(19) United States
(12) Patent Application Publication
(10) Pub. No.: US 2008/0261895 A1
(43) Pub. Date: Oct. 23, 2008

(54) Isolated hydroxy and n-oxide metabolites and derivatives of O-desmethylvenlafaxine and methods of treatment

(75) Inventors: Matthew J. Hoffmann, Pottstown, PA (US); William DeMaio, Collegeville, PA (US); Jim Wang, Malvern, PA (US); John W. Ullrich, Downingtwon, PA (US)

Correspondence Address: WYETH/FINNEGAN HENDERSON, LLP, 901 NEW YORK AVENUE, NW, WASHINGTON, DC, 20001-4413, US

(73) Assignee: Wyeth

(21) Appl. No.: 11/976,413

(22) Filed: Oct. 24, 2007

Related U.S. Application Data
(60) Provisional application No. 60/854,063, filed on Oct. 25, 2006.

(51) Int. Cl. A61K 31/7032; A61K 31/138; C07C 215/46; A61K 31/14; C07C 211/63; C07H 15/20

(52) U.S. Cl. 514/25; 564/319; 514/648; 536/17.9; 564/283; 514/643

(57) ABSTRACT

The present invention provides novel isolated compounds characterized as metabolites or derivatives of desmethylvenlafaxine including hydroxy-DV metabolites, hydroxy-DV-glucuronide metabolites, N-oxide-DV metabolites, and benzyl-hydroxy-DV metabolites. The invention includes pharmaceutical compositions comprising any of the metabolites or derivatives of the invention in combination with a pharmaceutically acceptable carrier or excipient. The invention also includes a method of treating at least one central nervous system disorder in a mammal comprising providing to a mammal in need thereof an effective amount of the compounds of the invention.

### BACKGROUND

Venlafaxine, chemically named 1-[2-(dimethylamino)-1-(4-methoxyphenyl)ethyl]cyclohexanol, has been shown to be a potent inhibitor of monoamine neurotransmitter uptake, a mechanism associated with clinical antidepressant activity. Due to its novel structure, venlafaxine has a mechanism of action unrelated to other available antidepressants, such as the tricyclic antidepressants desipramine, nostriptyline, protriptyline, imipramine, amitryptyline, trimipramine and doxepin.

It is believed that venlafaxine's mechanism of action is related to potent inhibition of the uptake of the monoamine neurotransmitters serotonin and norepinephrine. To a lesser degree, venlafaxine also inhibits dopamine reuptake, but it has no inhibitory activity on monoamine oxidase. O-desmethylvenlafaxine, venlafaxine's major metabolite in humans, exhibits a similar pharmacologic profile. Venlafaxine's ability to inhibit norepinephrine and serotonin (5-HT) uptake has been predicted to have an efficacy which rivals or surpasses that of tricyclic antidepressants. Montgomery, S. A., Venlafaxine: A New Dimension in Antidepressant Pharmacotherapy, J. Clin. Psychiatry, 54(3):119 (1993).

In contrast to classical tricyclic antidepressant drugs, venlafaxine has virtually no affinity for muscarinic, histaminergic or adrenergic receptors in vitro. Pharmacologic activity at these receptors is associated with the various anticholinergic, sedative and cardiovascular effects seen with the tricyclic antidepressant drugs.

Venlafaxine is disclosed in U.S. Pat. No. 4,535,186 (Husbands et al.) and has been previously reported to be useful as an antidepressant.

O-desmethylvenlafaxine (“DV”), chemically named 1-[2-(dimethylamino)-1-(4-phenol) ethyl]-cyclohexanol, is a major metabolite of venlafaxine and has been shown to inhibit norepinephrine and serotonin uptake. Klamerus, K. J. et al., “Introduction of the Composite Parameter to the Pharmacokinetics of Venlafaxine and its Active O-Desmethyl Metabolite”, J. Clin. Pharmacol. 32:716-724 (1992). A particularly useful novel salt form of O-desmethyl venlafaxine with unique properties, O-desmethylvenlafaxine succinate (“DVS”), was disclosed in U.S. Pat. No. 6,673,838 (Hadfield et al.).

Previously, only a limited understanding of the metabolites formed from venlafaxine and O-desmethylvenlafaxine, whether in their free base or salt forms, existed. Therefore, while some information on the metabolic products of venlafaxine was known, see Howell, S. R. et al., “Metabolic Disposition of ¹⁴C-Venlafaxine in Mouse, Rat, Dog, Rhesus Monkey and Man,” Xenobiotica 23(4):349359 (1993), the prior art lacked a complete understanding of all of the metabolic products and the activities therefore. The inventors now have a more complete understanding of the metabolites produced and the end uses therefor.

### SUMMARY OF THE INVENTION

The present invention provides novel isolated compounds that were characterized as metabolites or derivatives of DV, their corresponding pharmaceutical compositions, and methods of treatment.

Specifically, the present invention includes an isolated Hydroxy-DV metabolite or derivative of the formula

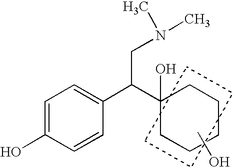

wherein
a hydroxy group is attached to one 2-position (ortho-position) or 3-position (meta-position) carbon on the cyclohexyl ring as shown by the dashed-line box;
and pharmaceutically acceptable salts thereof. In one embodiment, the isolated DV metabolite is a 2-Hydroxy-DV metabolite. In another embodiment, the isolated DV metabolite is a 3-Hydroxy-DV metabolite.

The invention also includes an isolated Hydroxy-DV glucuronide metabolite or derivative of the formula

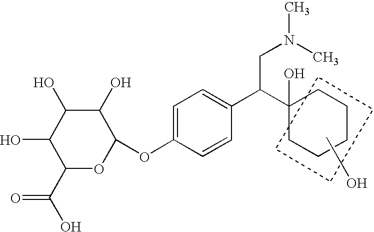

wherein a hydroxy group is attached to one 2-position (ortho), 3-position (meta), or 4-position (para) carbon on the cyclohexyl ring as shown by the dashed-line box;
and pharmaceutically acceptable salts thereof. In one embodiment, the isolated DV metabolite is a 2-Hydroxy-DV glucuronide metabolite. In another embodiment, the isolated DV metabolite is a 3-Hydroxy-DV glucuronide metabolite. In a third embodiment, the isolated DV metabolite is a 4-Hydroxy-DV glucuronide metabolite.

The invention further includes an isolated N-Oxide DV metabolite or derivative of the formula

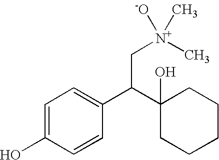

and pharmaceutically acceptable salts thereof.

The invention further includes isolated Benzyl Hydroxy-DV metabolites or derivatives of the formula

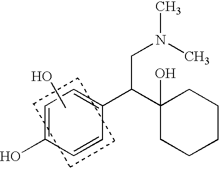

wherein a hydroxy group is attached to one 2-position or 3-position carbon on the benzyl;
and pharmaceutically acceptable salts thereof. In one embodiment, the isolated DV metabolite is 2-Benzyl Hydroxy-DV. In another embodiment, the isolated DV metabolite is 3-Benzyl Hydrozy-DV.

Likewise, the invention includes pharmaceutical compositions comprising any of the metabolites or derivatives of the invention in combination with a pharmaceutically acceptable carrier or excipient. It includes a method of treating at least one central nervous system disorder in a mammal comprising providing to a mammal in need thereof an effective amount of the compounds of the invention.

### BRIEF DESCRIPTION OF THE FIGURES

FIG. 1 illustrates novel isolation compounds characterized as metabolites of DVS. FIG. 1(A) illustrates four unique hydroxyl-DV compounds. The —OH group on the cyclohexanol ring may be at any of the positions shown within the dashed box. FIG. 1(B) illustrates four unique hydroxyl-DV glucuronides. The —OH group on the cyclohexanol ring may be at any of the positions shown within the dashed box. FIG. 1(C) illustrates an N-oxide DV compound. FIG. 1(D) illustrates a benzyl hydroxy-DV compound. The —OH group on the benzyl ring may be at any of the positions shown within the dashed box.

FIG. 2 shows a method of synthesizing 2-hydroxy DV compounds.

FIG. 3 provides a method of synthesizing 2-hydroxy-DV glucuronides.

FIG. 4 illustrates a method of synthesizing N-oxide DV compounds.

FIG. 5 illustrates a method of synthesizing a benzyl hydroxy DV.

FIG. 6 provides representative radiochromatograms following a single oral (20 mg/kg) administration of DVS to rats. FIG. 5(A) shows male plasma 1 hour post-dose. FIG. 5(B) shows male urine collected 0-8 hours post-dose. FIG. 5(C) shows male feces collected 8-24 hours post-dose.

FIG. 7 illustrates the proposed fragmentation scheme and the product ion spectrum of m/z 264 for DVS.

FIG. 8 shows proposed fragmentation scheme and the product ion spectrum of m/z 280 for M6 in rats. Throughout the specification and drawings, the letter “M” followed by a number refers to a metabolite product as described herein.

FIG. 9 provides the proposed fragmentation scheme and the product ion spectrum of m/z 440 for M7 in rats.

FIG. 10 shows the proposed fragmentation scheme and the product ion spectrum of m/z 250 for M10 in rats.

FIG. 11 shows the proposed fragmentation scheme and the product ion spectrum of [m+h]⁺ (m/z 250) for synthetic N,O-didesmethylvenlafaxine.

FIG. 12 provides the proposed fragmentation scheme and the product ion spectrum of m/z 426 for M13 in rats.

FIG. 13 provides proposed fragmentation scheme and the product ion spectrum of m/z 280 for N-oxide DV in rats.

FIG. 14 shows representative radiochromatogram metabolite profiles following a single oral (30 mg/kg) administration of DVS to dogs (a) plasma 1 hour post-dose, (b) urine collected 8-24 hours post-dose and (c) feces collected 0-24 hours post-dose.

FIG. 15 provides the proposed fragmentation scheme and the product ion spectrum of m/z 280 for M6 in dogs.

FIG. 16 shows the proposed fragmentation scheme and the product ion spectrum of m/z 440 for M7 in dogs.

FIG. 17 shows the proposed fragmentation scheme and the product ion spectrum of m/z 280 for M9 in dogs.

FIG. 18 provides the proposed fragmentation scheme and the product ion spectrum of m/z 250 for M10 in dogs.

FIG. 19 shows the proposed fragmentation scheme and the product ion spectrum of m/z 456 for M12 in dogs.

FIG. 20 shows the proposed fragmentation scheme and the product ion spectrum of m/z 426 for M13 in dogs.

FIG. 21 provides the proposed fragmentation scheme and the product ion spectrum of m/z 236 for M14.

FIG. 22 shows the proposed fragmentation scheme and the products of [m+h]⁺ (m/z 236) mass spectrum for synthetic N,N,O-tridesmethylvenlafaxine.

FIG. 23 shows the proposed fragmentation scheme and the product ion spectrum of m/z 280 for N-oxide DV in dogs.

### DETAILED DESCRIPTION OF THE INVENTION

### I. Compounds of the Invention

A. Isolated DV Metabolites and Derivatives

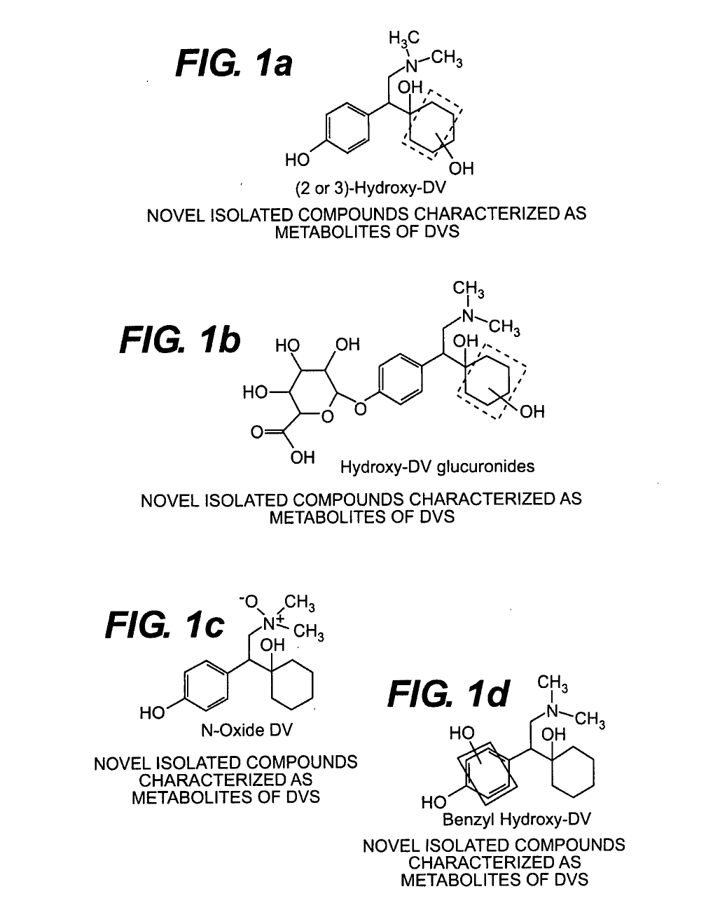

The present invention relates to newly identified metabolites and derivatives of DV expected to have beneficial properties. While some of the compounds are natural metabolites (those produced by enzymatic and other reactions in the body and in models therefor), others are related structures (derivatives) that are expected to exhibit substantially similar activity. FIG. 1 shows the structures of these compounds.

As shown in FIG. 1(A), the (2 or 3)-hydroxy-DV compounds are hydroxylated DV derivatives with the hydroxyl group attached on the cyclohexyl ring at one of the 2-position or 3-position carbons. The 2- and 3-position carbons are those within the dashed-line box in FIG. 1(A). There are eight total potential sites of attachment at the 2- and 3-position carbons (two on each carbon), however, due to symmetry, the sets of 2-position and 2-position carbons on the ring yield four distinct compounds. Therefore, the hydroxy group may attach to either of two positions on a 2-position carbon or either position on a 3-position carbon.

DV metabolites hydroxylated at any of the 2-, 3-, or 4-positions on the cyclohexyl ring may be glucuronidated to form cyclohexyl hydroxy-DV glucuronides, shown in FIG. 1(B). The hydroxy group may attach to any of the carbons within the dashed-line box.

FIG. 1(C) shows N-oxide DV, a DVS derivative with an oxygen at the nitrogen on the dimethyl amine group.

FIG. 1(D) shows benzyl hydroxy DV, a DVS metabolite or derivative with a hydroxy group attached to either the 2-position or 3-position carbon on the benzyl ring.

This application provides figures showing the structure of each compound, information on the compound as a metabolic product of DV, isolation, and/or synthesis, as well as expected activity for each compound.

1. Compounds Characterized from In Vivo Rat Experiments

The metabolism of DVS was investigated in rats following a single oral administration of 20 mg/kg (measured as amount of free base). DVS was extensively and rapidly metabolized in the rat, primarily to O-desmethylvenlafaxine O-glucuronide (DV glucuronide). DV glucuronide was the predominant drug-related compound in all plasma and urine samples analyzed.

M1-M6, six distinct hydroxyl-metabolites, were detected by LC/MS and in some samples by radiochromatography. In these metabolites, the hydroxyl group attaches at the 2-, 3-, and 4-positions on the cyclohexanol ring, yielding six distinct compounds, M1-M6. The glucuronides of these hydroxy DV metabolites were not observed in rats. N-oxide DV was observed via LC/MS in rat plasma, urine, and feces. Additional metabolites were also observed.

2. Compounds Characterized from In Vivo Dog Experiments

The metabolism of DVS in beagle dogs was determined following a single oral administration of 30 mg/kg (free base). DVS was extensively and rapidly metabolized in dogs. DV glucuronide was the most abundant metabolite detected by radiochromatography of urine and plasma samples.

Compounds M1-M6 were observed via LC/MS in plasma, urine, and feces. Compounds M11 and M12 were observed in urine (via radiochromatography and LC/MS). N-Oxide DV compounds were observed in plasma (via LC/MS), urine (via LC/MS), and feces (via radiochromatography and LC/MS). Additional metabolites were also observed.

In summary, DVS was rapidly and extensively metabolized to a number of metabolites in dogs. The most abundant metabolite detected was DV O-glucuronide. The metabolites observed in the current study were similar to those observed in rat plasma, urine, and feces following oral administration, with a greater number of metabolites being observed in beagle dogs.

B. Activity

The compounds of the present invention were detected as metabolites or derivatives of DVS, and are believed to exhibit a type of activity similar to that of venlafaxine and DVS. The hydroxy-DV glucuronides are believed to act as pro drugs, with the glucuronide being cleaved in vivo prior to activity. Cleavage of the glucuronide may occur via either the action of β-glucuronidase, which may be particularly active in the gastrointestinal tract, or under acidic conditions, such as those in the stomach. The hydroxyl-DV and N-oxide DV compounds are expected to be active in their current form. The compounds of the present invention may be tested for specific biological activities using receptor assay binding studies and in vivo metabolic and efficacy studies, which are well known in the art. See Example 5.

C. Synthesis

1. Syntheses of Free Base Compounds

The compounds of the present invention can be prepared using the methods described below, together with synthetic methods known in the synthetic organic arts or variations of these methods by one skilled in the art. See, generally, Comprehensive Organic Synthesis, “Selectivity, Strategy & Efficiency in Modern Organic Chemistry”, ed., I. Fleming, Pergamon Press, New York (1991); Comprehensive Organic Chemistry, “The Synthesis and Reactions of Organic Compounds”, ed. J. F. Stoddard, Pergamon Press, New York (1979). Suitable methods include, but are not limited to, those outlined below.

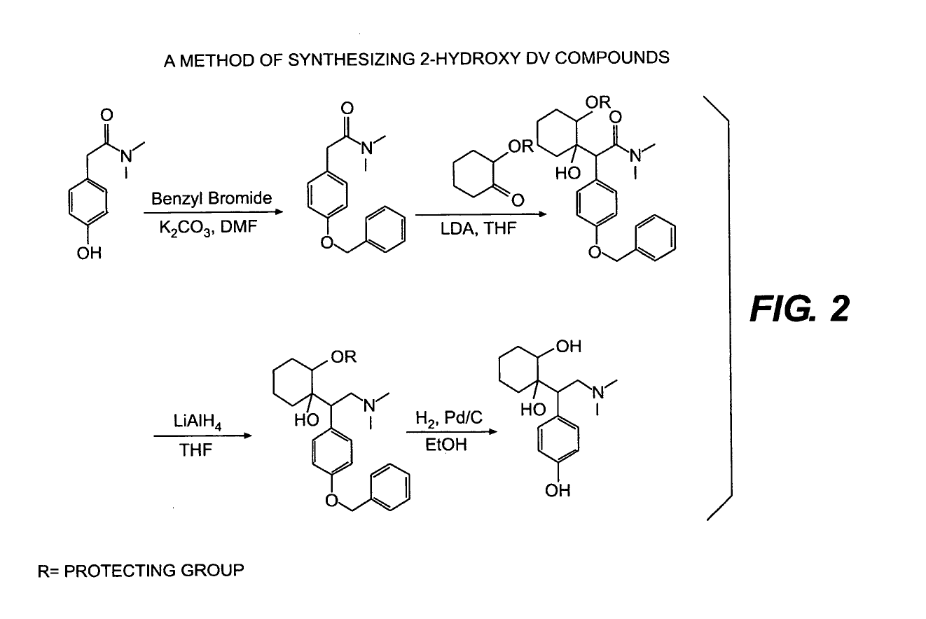

FIG. 2 provides one method for the synthesis of 2-hydroxy DV compounds of the invention. In the first step of this synthesis, 4-(dimethylcarbamoylmethyl)phenol is protected with a benzyl group. The benzyl bromide protecting group is well suited for use in the method of synthesizing the compounds of the invention because of its ease of removal during the final step. However, other protecting groups may be used.

In the second step, an acidic solution of a protected 2-hydroxy cyclohexanone (protected at the hydroxy) is added under appropriate using lithium diisopropylamide as a reagent. Suitable protecting groups are known in the art, and include benzyl-, trimethylsilyl-, and tert-butyl-dimethylsilyl-groups.

In the third step, the ketone is removed using lithium aluminum hydroxide. Alternatively, the ketone may be removed using sodium borohydride. The final step shows removal of the protecting groups. A similar method can be used for synthesis of the 3-hydroxy DV compounds, using the appropriate protected 3-substituted cyclohexanone. In addition, this method can be used to prepare 4-hydroxy DV compounds using an appropriate protected 4-substituted cyclohexanone.

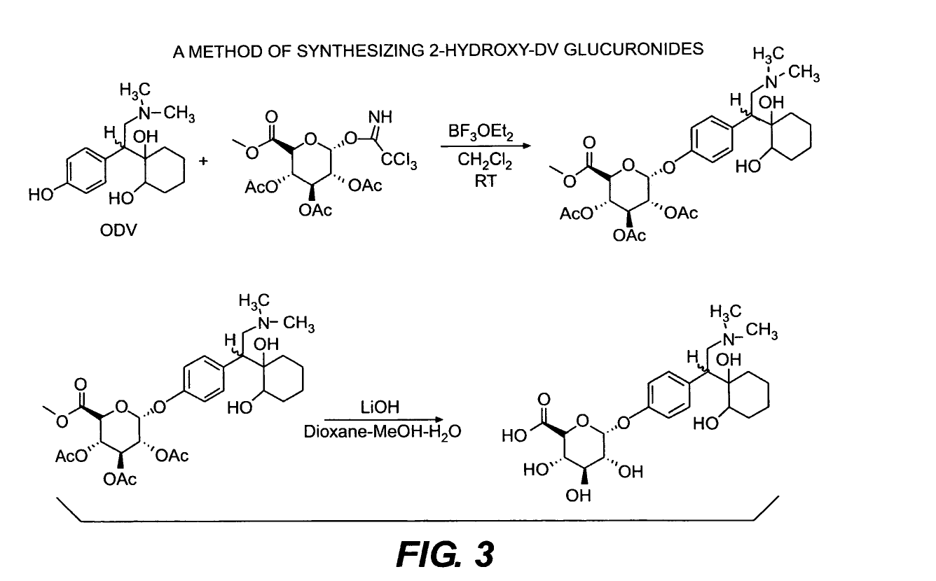

FIG. 3 provides one method for the synthesis of the hydroxy DV glucuronides. In this method, an appropriate hydroxy-DV compound is coupled to a trichloroimidate of glucuronide, as shown in the figure.

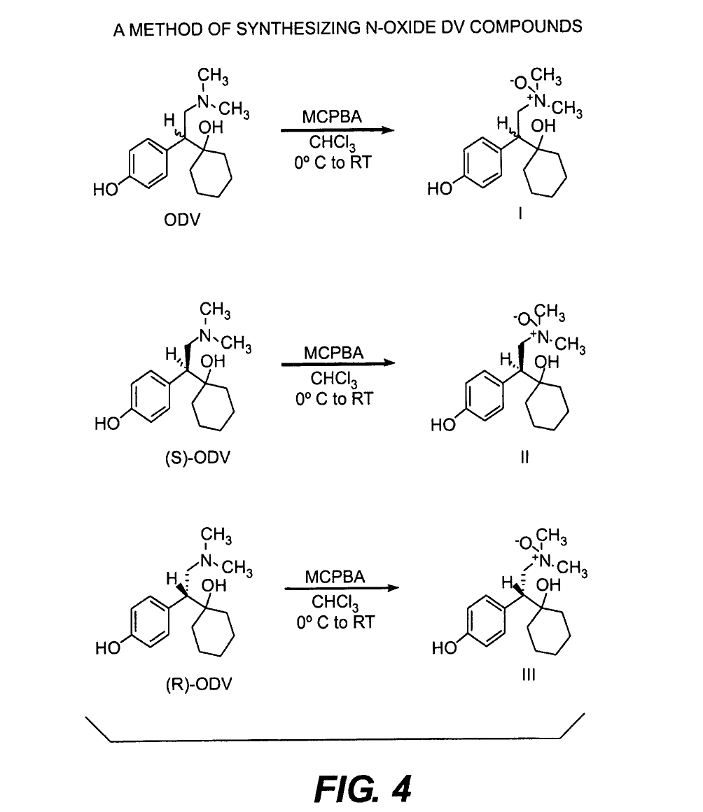

FIG. 4 provides one method for the synthesis of N-oxide DV compounds. In this method, N-oxide DV is prepared by oxidizing O-desmethylvenlafaxine with 3-chloroperoxybenzoic acid (MCPBA).

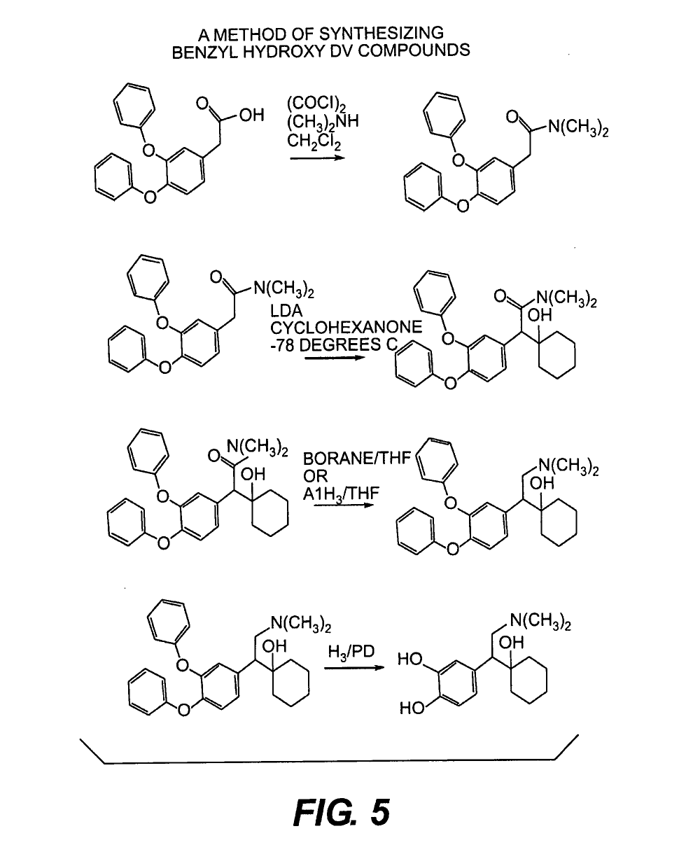

Benzyl hydroxy DV compounds can be prepared following the procedures outlined in Yardley, J P et al., “2-Phenyl-2-(1-hydroxycycloalkyl)ethylamine Derivatives: Synthesis and Antidepressant Activity,” Journal of Medicinal Chemistry 33(10): 2899-905 (1990). One of skill in the art would be able to adapt the synthetic schemes for the preparation of other structures depicted in Yardley to synthesize both the 2-benzyl hydroxy DV compounds and the 3-hydroxy DV compounds in light of the present discovery that such compounds are desired. For example, starting with (3,4-bis-benzyloxy-phenyl)-acetic acid, a 3-substituted benzyl hydroxy DV can be prepared as shown in FIG. 5. As another example, (2,4-Bis-benzyloxy-phenyl)-acetic acid could be used to prepare a 2-substituted benzyl hydroxy DV compound. Alternatively, following procedures in Yardley, (2,4-Bis-benzyloxy-phenyl)-acetonitrile and (3,4-Bis-benzyloxy-phenyl)-acetonitrile could be used to prepare the corresponding 2- and 3-substituted benzyl hydroxy DV compounds.

2. Syntheses of Salts

The compounds of the present invention can have utility in both their free base and salt forms. The pharmaceutically acceptable acid addition salts of the basic compounds of this invention are formed conventionally by reaction of the free base with an equivalent amount of any acid which forms a non-toxic salt. Illustrative acids are either inorganic or organic, including hydrochloric, hydrobromic, fumaric, maleic, succinic, sulfuric, phosphoric, tartaric, acetic, citric, oxalic, benzenesulfonic, benzoic, camphorsulfonic, ethenesulfonic, gluconic, glutamic, isethionic, lactic, malic, mandelic, methanesulfonic, mucic, nitric, pamoic, pantothenic, p-toluenesulfonic and similar acids. For parenteral administration, water soluble salts may be used, although either the free base or the pharmaceutically acceptable salts are applicable for oral or parenteral administration of the compounds of this invention.

3. Stereochemistry

The compounds of the present invention exist as enantiomers and this invention includes racemic mixtures as well as stereoisomerically pure forms of the compounds of the invention (both the R-enantiomer and the S-enantiomer), unless otherwise indicated.

D. Isolation

Alternatively, the compounds of the present invention can be isolated from plasma, urine, or fecal samples containing the compound, or from an in vitro system containing the compound using techniques known in the art. Specifically, the compounds may be isolated using preparatory-scale HPLC (prep-HPLC) under conditions that lead to a separation of the individual metabolites, for example, using a linear gradient of two mobile phases, A and B, wherein mobile phase A may be 10 mM ammonium acetate, pH 5.5, and mobile phase B may be acetonitrile, at a flow rate leading to separation, as described in Examples 1-2.

Such isolated compounds may be in purified form or may be in substantially purified form, meaning that they are removed from their natural environment. Substantially pure compounds include compounds that are 99%, 98%, 97%, 96%, 95%, 94%, 93%, 92%, 91%, 90%, 85%, 80%, 75%, 70%, 65% pure.

E. Pharmaceutical Dosage Forms

Pharmaceutical compositions containing the compounds of this invention represent an additional aspect of this invention. The active ingredients can be compounded into any of the usual oral dosage forms including tablets, capsules and liquid preparations such as elixirs and suspensions containing various coloring, flavoring, stabilizing and flavor masking substances. For compounding oral dosage forms, the active ingredient can be mixed with various conventional tabletting materials such as starch, calcium carbonate, lactose, sucrose and dicalcium phosphate to aid the tabletting or capsulating process. Magnesium stearate, as an additive, provides a useful lubricant function when desired. The active ingredients can be dissolved or suspended in a pharmaceutically acceptable sterile liquid carrier, such as sterile water, sterile organic solvent or a mixture of both. A liquid carrier may be one suitable for parenteral injection. Where the active ingredient is sufficiently soluble it can be dissolved in normal saline as a carrier; if it is too insoluble for this it can often be dissolved in a suitable organic solvent, for instance aqueous propylene glycol or polyethylene glycol solutions. Aqueous propylene glycol containing from 10 to 75% of the glycol by weight is generally suitable. In other instances other compositions can be made by dispersing the finely-divided active ingredient in aqueous starch or sodium carboxymethyl cellulose solution, or in a suitable oil, for instance arachis oil. Liquid pharmaceutical compositions which are sterile solutions or suspensions can be utilized by intramuscular, intraperitoneal or subcutaneous injection.

The compounds of the present invention can be combined with a pharmaceutical carrier or excipient (e.g., pharmaceutically acceptable carriers and excipients) according to conventional pharmaceutical compounding technique to form a pharmaceutical composition or dosage form. Suitable pharmaceutically acceptable carriers and excipients include, but are not limited to, those described in Remington's, The Science and Practice of Pharmacy, (Gennaro, A. R., ed., 19th edition, 1995, Mack Pub. Co.). The phrase “pharmaceutically acceptable” refers to additives or compositions that are physiologically tolerable and do not typically produce an allergic or similar untoward reaction, such as gastric upset, dizziness and the like, when administered to an animal, such as a mammal (e.g., a human). For oral liquid pharmaceutical compositions, pharmaceutical carriers and excipients can include, but are not limited to water, glycols, oils, alcohols, flavoring agents, preservatives, coloring agents, and the like. Oral solid pharmaceutical compositions may include, but are not limited to starches, sugars, microcrystalline cellulose, diluents, granulating agents, lubricants, binders and disintegrating agents. The pharmaceutical composition and dosage form may also include venlafaxine, O-desmethylvenlafaxine, or salts thereof as discussed above.

Dosage forms include, but are not limited to tablets, troches, lozenges, dispersions, suspensions, suppositories, ointments, cataplasms, pastes, powders, creams, solutions, capsules (including encapsulated spheroids), and patches. The dosage forms may also include immediate release as well as formulations adapted for controlled, sustained, extended, or delayed release. Tablets and spheroids may be coated by standard aqueous and nonaqueous techniques as required.

Pharmaceutical composition may be in unit dosage form, e.g. as tablets or capsules. In such form, the composition is sub-divided in unit doses containing appropriate quantities of the active ingredient; the unit dosage forms can be packaged compositions, for example, packeted powders or vials or ampoules. The unit dosage form can be a capsule, cachet or tablet itself, or it can be the appropriate number of any of these in package form. The quantity of the active ingredient in a unit dose of composition may be varied or adjusted according to the particular need and the activity of the active ingredient.

### II. Methods of Treatment

A. Diseases that may be treated

The methods of the present invention involve administering to a mammal in need thereof an effective amount of one or more of the compounds of the present invention.

The compounds of the present invention are believed to have activity of a type similar to that of venlafaxine and O-desmethylvenlafaxine. The hydroxy-DV glucuronides may act as a pro drugs, losing the glucuronide appendage in vivo and forming the corresponding hydroxyl-DV compounds. Cleavage of the glucuronide may occur via either the action of β-glucuronidase, which may be particularly active in the gastrointestinal tract, or under acidic conditions, such as those in the stomach. The remaining compounds are expected to have activity in their current forms.

As described in Reviews in Contemporary Pharmacology, Volume 9(5) page 293-302 (1998), O-desmethyl-venlafaxine has the pharmacological profile shown in Table 1.

TABLE 1
PHARMACOLOGICAL PROFILE FOR
O-DESMETHYLVENLAFAXINE

| Effect (in vivo) | |
|---|---|
| Reversal of Reserpine-Induce | 3 |
| Hypothermia | |
| (minimum effect; mg&g i.p.): | |
| Effect (in vitro) | |
| Inhibition of amine reuptake (IC50; | |
| uM): | |
| Norepinephrine | 1.16 |
| Serotonin | 0.18 |
| Dopamine | 13.4 |
| Affinity for Various Neuroreceptors | |
| (% inhibition at 1 uM): | |
| D2 | 6 |
| s Cholinergic | 7 |
| Adrenergic a | 0 |
| Histamine Hi | 0 |
| Opiate | 7 |

Thus, compounds, compositions and methods of the present invention may be used to treat patients suffering from or susceptible to at least one central nervous system disorder including, but not limited to depression (including but not limited to major depressive disorder, bipolar disorder and dysthymia), fibromyalgia, anxiety, panic disorder, agorophobia, post traumatic stress disorder, premenstrual dysphoric disorder (also known as premenstrual syndrome), attention deficit disorder (with and without hyperactivity), obsessive compulsive disorder (including trichotillomania), social anxiety disorder, generalized anxiety disorder, autism, schizophrenia, obesity, anorexia nervosa, bulimia nervosa, Gilles de la Tourette Syndrome, vasomotor flushing, cocaine and alcohol addiction, sexual dysfunction (including premature ejaculation), borderline personality disorder, chronic fatigue syndrome, incontinence (including fecal incontinence, overflow incontinence, passive incontinence, reflex incontinence, stress urinary incontinence, urge incontinence, urinary exertional incontinence and urinary incontinence), pain (including but not limited to migraine, chronic back pain, phantom limb pain, central pain, neuropathic pain such as diabetic neuropathy, and postherpetic neuropathy), Shy Drager syndrome, Raynaud's syndrome, Parkinson's Disease, epilepsy, and others. Compounds and compositions of the present invention can also be used for preventing relapse or recurrence of depression, including continuing treatment of a patient who previously had depression and is in a state of remission; to treat cognitive impairment; for the inducement of cognitive enhancement and/or enhanced mood in patient suffering from senile dementia, Alzheimer's disease, memory loss, amnesia and amnesia syndrome; and in regimens for cessation of smoking or other tobacco uses. Additionally, compounds and compositions of the present invention can be used for treating hypothalamic amenorrhea in depressed and non-depressed human females.

B. Administration and Dosage

This invention provides methods of treating, preventing, inhibiting or alleviating each of the maladies listed above in a mammal, including a human, the methods comprising administering an effective amount of a compound of the invention to a mammal in need thereof. An effective amount is an amount sufficient to prevent, inhibit, or alleviate one or more symptoms of the aforementioned conditions.

The dosage amount useful to treat, prevent, inhibit or alleviate each of the aforementioned conditions will vary with the severity of the condition to be treated and the route of administration. The dose and dose frequency will also vary according to age, body weight, response and past medical history of the individual human patient. In general, the recommended daily dose range for the conditions described herein include from 10 mg to 1000 mg per day of a compound of the present invention. Other appropriate dosages include from 50 mg to 800 mg per day, from 75 mg to 600 mg per day, from 100 mg to 500 mg per day, and from 150 mg to 300 mg per day, and 200 mg per day. Specific dosages include all of the endpoints listed above. Dosage is described in terms of the free base, and not in terms of any particular pharmaceutically acceptable salt. In managing the patient, the therapy may be initiated at a lower dose and increased if necessary. Dosages for non-human patients can be adjusted accordingly by one skilled in the art.

The compounds of the present invention may also be provided in combination with venlafaxine, O-desmethylvenlafaxine, DVS, or other pharmaceutically acceptable salts thereof. The compounds of the present invention may also be provided with other known psychiatrically-active compounds, such as other antidepressants or antianxiety drugs, hormonal treatments, pain medications, and other therapies.

Any suitable route of administration can be employed for providing the patient with an effective amount of the compounds of interest. For example, oral, mucosal (e.g. nasal, sublingual, buccal, rectal or vaginal), parental (e.g. intravenous or intramuscular), transdermal, and subcutaneous routes can be employed.

The following examples are illustrative but are not meant to be limiting of the present invention.

### EXAMPLES

### Example 1

### Metabolism of [¹⁴C]DVS in Sprague Dawley Rats Following a Single Oral Administration

Six hydroxy DV compounds and N-oxide DV compounds, as well as other compounds, were detected in the metabolic profiles for [¹⁴C]DVS in urine, feces, and plasma following a single oral gavage dose in male and female rats as described below.

Radiolabeled [¹⁴C]DVS (Batch #CFQ13003, [cyclohexyl-1-¹⁴C]DVS) was supplied by Amersham Biosciences (Buckinghamshire, UK). Unlabeled DVS (Batch RB1636; free base 65.2%) was received from Wyeth Research, Rouses Point, N.Y. The average molecular weight of DVS is 381.5, with O-desmethylvenlafaxine, accounting for 69.0% by weight. The specific activity of [¹⁴C]DVS (bulk drug) was 144 μCi/mg (209 μCi/mg for the free base) and the radiopurity of the free base was 99.3%, as determined by HPLC using radiometric detection.

Water for preparation of the oral dosing solution was obtained from EM Science (Gibbstown, N.J.). Methylcellulose and polysorbate 80 were received from Sigma Chemical Co. (St. Louis, Mo.) and Mallinckrodt Baker (Phillipsburg, N.J.), respectively. The liquid scintillation cocktail used in counting the radioactivity in urine and plasma samples, fecal homogenate extracts and the dosing solution aliquots was Ultima Gold™ (Perkin Elmer, Wellesley, Mass.).

A model 307 Tri-Carb Sample Oxidizer, equipped with an Oximate-80 Robotic Automatic Sampler (Perkin Elmer), was used for combustion of blood and fecal samples. PermaFluor® E⁺ liquid scintillation cocktail (Perkin Elmer), Carbosorb® E (Perkin Elmer) carbon dioxide absorber and HPLC grade water were used to trap radioactive carbon dioxide generated by combustion of the samples in the oxidizer. Fecal homogenates and blood samples were transferred to combusto-cones and cover pads (Perkin Elmer) for combustion.

Sprague Dawley rats (12 male and 6 female), weighing between 0.311 and 0.345 kg for males and between 0.263 and 0.311 kg for females at the time of dosing, were used. Animals were given food and water ad libitum. For ease of reporting, the animals were designated numbers 001M through 012M for the male rats and 001F through 006F for the female rats. Three animals from the last time point, for each sex, were housed individually in metabolism cages for the collection of urine and feces. The other animals were housed individually in standard cages.

The oral dosing solution was prepared by combining 86.4 mL of 3.0 mg/mL (2.0 mg/mL, free base) unlabeled DVS solution with 3.6 mL of 4.3 mg/mL (3.0 mg/mL free base) [¹⁴C]DVS solution. Solutions were prepared in 0.25% polysorbate 80, 0.5% methylcellulose in water. The radiochemical purity, specific activity and concentration of [¹⁴C]DVS (bulk drug and dosing solution) were determined using HPLC with radiometric detection. Aliquots of the dosing solution were taken pre-, mid-, and post-dose for the determination of specific activity and radioactivity concentrations of dosing solution.

The target dose for each animal was 30 mg/kg (free base; 3.0 mg/mL, 10 mL/kg, 250 μCi/kg) [¹⁴C]O-desmethylvenlafaxine via oral gavage.

Whole blood (approximately 5 mL) was collected by cardiac puncture into heparinized tubes at the appropriate time points (1, 4, 8, and 24 hours for male rats, and 1 and 8 hours for female rats, N=3 for each sex at each time point). Triplicate aliquots (200 μL) of whole blood were placed into combusto-cones, weighed and allowed to air dry. These samples were oxidized. The remaining blood was centrifuged at 5000×g and 4° C. for 10 minutes (Model Legend RT centrifuge, Sorvall). The resulting plasma was transferred to fresh tubes and triplicate aliquots (100 μL) were analyzed for radioactivity content. The remaining plasma was stored at −70° C. until metabolite analysis.

Urine and feces were collected separately on dry ice from three animals per sex. Collections were from 0-8 and 8-24 hours for male rats and 0-8 hours for female rats. Fecal samples were homogenized in approximately five volumes (v/w) of water. Aliquots of approximately 0.4 grams of the homogenate were placed into combusto-cones, weighed and allowed to air dry. These samples were then oxidized. The remaining urine samples and fecal homogenates were stored at −70° C. until metabolite analysis.

Blood samples and fecal homogenates were oxidized in a Model 307 Tri-Carb sample oxidizer, using Carbosorb® E (6 mL) as trapping agent and PermaFluor® E⁺ (10 mL) as scintillant. Oxidation efficiency was determined by oxidation of C-Spec-Chec (Perkin Elmer), a standard of known radioactivity, and determined to be 98.7%. The background reading (average of control blood or fecal samples) was subtracted from each sample reading. Aliquots of urine and plasma were analyzed directly following the addition of 10 mL of Ultima Gold™ scintillation fluid.

All radioactivity determinations were made using a Tri-Carb Model 3100TR liquid scintillation counter (Perkin Elmer) with an Ultima Gold™ or toluene standard curve. Counts per minute (CPM) were converted to disintegrations per minute (DPM) by use of external standards of known radioactivity. The quench of each standard was determined by the transformed spectral index of an external radioactive standard (tSIE). The lower limits of detection were defined as twice background.

### Plasma Metabolite Samples

Plasma samples collected at 1, 4 and 8 hours post-dose were analyzed for metabolite profiles. Aliquots of 0.5 mL plasma were mixed with an equal volume of acetonitrile, placed on ice for approximately 10 minutes, and then centrifuged at 3500 rpm and 4° C. in a Sorvall Super 21 centrifuge for 10 minutes. The supernatant fluid was transferred to a clean tube. The supernatant was analyzed for radioactivity. The supernatant was concentrated under a stream of nitrogen in a Turbo Vap (Zymark, Hopinkton, Mass.) to remove the acetonitrile. An aliquot of the aqueous residue was analyzed by HPLC for metabolite profiling. Selected samples were also analyzed by LC/MS to characterize the radioactive peaks.

The stability of [¹⁴C]DVS in rat plasma was determined. [¹⁴C]DVS (0.01 mg/mL, final concentration) was added to control rat plasma and incubated in a shaking water bath set to 37° C. Aliquots (0.5 mL) were removed at 0, 1, 4, 8 and 24 hours. Samples were extracted as described above and radiopurity assayed by HPLC analysis.

### Urine Metabolite Samples

All urine samples were analyzed for metabolite profiles. Aliquots of 0.5 mL urine were centrifuged at 3500 rpm and 4° C. in a Sorvall Super 21 centrifuge for 10 minutes. The supernatant was transferred to a fresh tube and analyzed for radioactivity content and by HPLC for profiling. Selected samples were also analyzed by LC/MS to characterize the radioactive peaks.

The stability of [¹⁴C]DVS in rat urine was determined. [¹⁴C]DVS (0.13 mg/mL, final concentration) was added to control rat urine and incubated in a shaking water bath set to 37° C. Aliquots (0.5 mL) were removed at 0, 1, 4, 8 and 24 hours. Samples were extracted as described above and radiopurity assayed by HPLC analysis.

### Fecal Metabolite Samples

Fecal homogenates collected from male rats between 8 and 24 hours post-dose were analyzed for metabolite profiles. Aliquots of approximately 1 gram of fecal homogenate were centrifuged at 3500 rpm and 4° C. in a Sorvall Super 21 centrifuge for 10 minutes. The supernatant was transferred to a clean tube. The residue was re-suspended with 1 mL of water:acetonitrile (1:1, v:v) and centrifuged as described above. The resulting supernatant was combined with the original supernatant and the residue re-suspended with 1 mL of acetonitrile. The suspension was centrifuged as described above, and the supernatants were combined and analyzed for radioactivity. The supernatants were then concentrated under a stream of nitrogen in a Turbo Vap to remove the acetonitrile. An aliquot of the aqueous residue was analyzed by HPLC for profiling. Selected samples were also analyzed by LC/MS to characterize the radioactive peaks.

### Sample Analysis

Chromatographic analyses were performed with a Waters Alliance model 2690 HPLC system (Waters Corp., Milford, Mass.). It was equipped with a built-in autosampler and was in-line with a model 2487 tunable UV detector, set to monitor 225 nm, and a FloOne β Model 525 radioactivity flow detector (Perkin Elmer) with a 250 μL LQTR flow cell. The flow rate of Ultima Flow M scintillation fluid was 1 mL/min, providing a mixing ratio of scintillation cocktail to mobile phase of 5:1. Separation of the metabolite peaks was accomplished on a Phenomenex Luna C18(2) column, 150×2.0 mm, 5 micron (Phenomenex, Torrance, Calif.), using a linear gradient of two mobile phases, A and B. Mobile phase A was 10 mM ammonium acetate, pH 6.0, and mobile phase B was acetonitrile. The flow rate was 0.2 mL/min. The mobile phase was delivered as shown in Table 2.

TABLE 2
CHROMATOGRAPHIC MOBILE PHASE DELIVERY
CONDITIONS.

| Time | Mobile phase A | Mobile phase B | Flow rate |
|---|---|---|---|
| (min) | (%) | (%) | (mL/min) |
| 0 | 95 | 5 | 0.2 |
| 30 | 85 | 15 | 0.2 |
| 40 | 85 | 15 | 0.2 |
| 41 | 5 | 95 | 0.5 |
| 55 | 5 | 95 | 0.5 |
| 56 | 95 | 5 | 0.5 |
| 62 | 95 | 5 | 0.5 |
| 63 | 95 | 5 | 0.2 |
| 65 | 95 | 5 | 0.2 |

MOBILE PHASE A = 10 MM AMMONIUM ACETATE IN WATER, PH 5.5.
MOBILE PHASE B = ACETONITRILE.

An Agilent Model 1100 HPLC system (Agilent Technologies, Wilmington, Del.) including an autosampler and diode array UV detector was used for LC/MS analysis. The UV detector was set to monitor 200 to 400 nm. Separations were accomplished on a 5 micron Phenomenex Luna C18(2) column, 150×2 mm (Phenomenex). The column temperature was 25° C. The mobile phases and gradient program were as follows.

The mass spectrometer used for metabolite characterization was a Micromass Q-TOF-2 quadrupole time-of-flight hybrid mass spectrometer (Micromass, Inc., Beverly, Mass.). The mass spectrometer was equipped with an electrospray ionization (ESI) interface and operated in the positive ionization mode. Settings for the mass spectrometer are listed in Table 3.

TABLE 3
MICROMASS Q-TOF-2 MASS
SPECTROMETER SETTINGS

| | |
|---|---|
| Capillary Voltage | 3.2 kV |
| Cone | 28 V |
| Source Block | 80° C. |
| Temperature | |
| Desolvation Temperature | 200° C. |
| Desolvation Gas Flow | 350 L/hr |
| Cone Gas Flow | 75 L/hr |
| CID Gas Inlet Pressure | 13-14 psig |

FloOne analytical software (version 3.65, Packard BioScience, Boston, Mass.) was utilized for data collection and analysis of the radioactive peaks. The computer program Microsoft Excel®97 was used to calculate means and standard deviations. MassLynx software (version 3.5) was used to analyze LC/MS data.

### Results

The radiochemical purity and specific activity of [¹⁴C]DVS (bulk compound), determined by HPLC with radiometric detection, were 99.3% and 209 μCi/mg (free base), respectively. The concentration, radiopurity and specific activity of [¹⁴C]O-desmethylvenlafaxine in the dosing solution were 2.05 mg/mL, 97.8% and 11.7 μCi/mg, respectively. Pre-, mid- and post-dose aliquots of the dosing solution had similar concentrations and purities. The mean administered dose of [¹⁴C]DVS was 19.9±0.24 mg/kg (free base). This dose deviated from the target dose of 30 mg/kg (free base) because the original weighing for the dose preparation did not take into account that DVS is the succinate salt of O-desmethylvenlafaxine (free base).

### Stability of [¹⁴C]DVS in Control Rat Urine and Plasma

[¹⁴C]DVS was stable at 37° C. for up to 24 hours in both control rat urine and control rat plasma. The radiopurity of [¹⁴C]DVS in rat plasma was greater than 98.9% at all time points, while in rat urine the radiopurity was greater than 99.5% at all time points.

### Blood to Plasma Partitioning

The concentrations of radioactivity in blood and plasma, and the blood to plasma partitioning are shown in Table 4. There were no significant differences in the concentration of radioactivity detected in blood or plasma between male and female rats. The mean plasma concentrations of total radioactivity in male rats were 11.0, 1.48, 0.89 and 0.07 μg equivalents/mL at 1, 4, 8 and 24 hour post-dose, respectively. For female rats, the mean plasma concentrations of total radioactivity were 9.90 and 0.92 μg equivalents/mL at 1 and 8 hour post-dose, respectively. At the 1, 4 and 8 hour time points, the blood to plasma ratio for radioactivity ranged between 0.59 and 0.67 in both sexes, while at the 24 hour time point the ratio was 0.99 in male rats.

TABLE 4
WHOLE BLOOD AND PLASMA RADIOACTIVITY
CONCENTRATIONS AND PARTITIONING OF THE RADIOACTIVITY
FOLLOWING A SINGLE ORAL (20 MG/KG) ADMINISTRATION OF [¹⁴C]DVS TO
RATS

| | | | | | | | | | Blood to |
|---|---|---|---|---|---|---|---|---|---|
| Sampling | Radioactivity in Whole Blood | | | | Radioactivity in Plasma | | | | Plasma |
| Time | (μg equivalents/mL) | | | | (μg equivalents/mL) | | | | Ratio |
| (hr/sex) | Individual Rats | | | Mean ± S.D. | Individual Rats | | | Mean ± S.D. | Mean ± S.D. |
| 1/M | 6.11 | 5.66 | 8.85 | 6.87 ± 1.50 | 10.4 | 8.90 | 13.7 | 11.0 ± 2.2 | 0.62 ± 0.03 |
| 1/F | 5.63 | 5.76 | 7.15 | 6.18 ± 0.74 | 9.61 | 8.74 | 11.3 | 9.90 ± 1.2 | 0.63 ± 0.04 |
| 4/M | 0.95 | 0.96 | 0.72 | 0.88 ± 0.12 | 1.59 | 1.62 | 1.22 | 1.48 ± 0.20 | 0.59 ± 0.00 |
| 8/M | 0.35 | 0.71 | 0.71 | 0.59 ± 0.18 | 0.53 | 1.10 | 1.05 | 0.89 ± 0.27 | 0.67 ± 0.01 |
| 8/F | 0.59 | 0.50 | 0.66 | 0.58 ± 0.07 | 0.86 | 0.82 | 1.08 | 0.92 ± 0.12 | 0.64 ± 0.05 |
| 24/M | 0.06 | 0.08 | 0.05 | 0.07 ± 0.01 | 0.07 | 0.08 | 0.05 | 0.07 ± 0.01 | 0.99 ± 0.04 |

### Plasma Metabolite Profiles

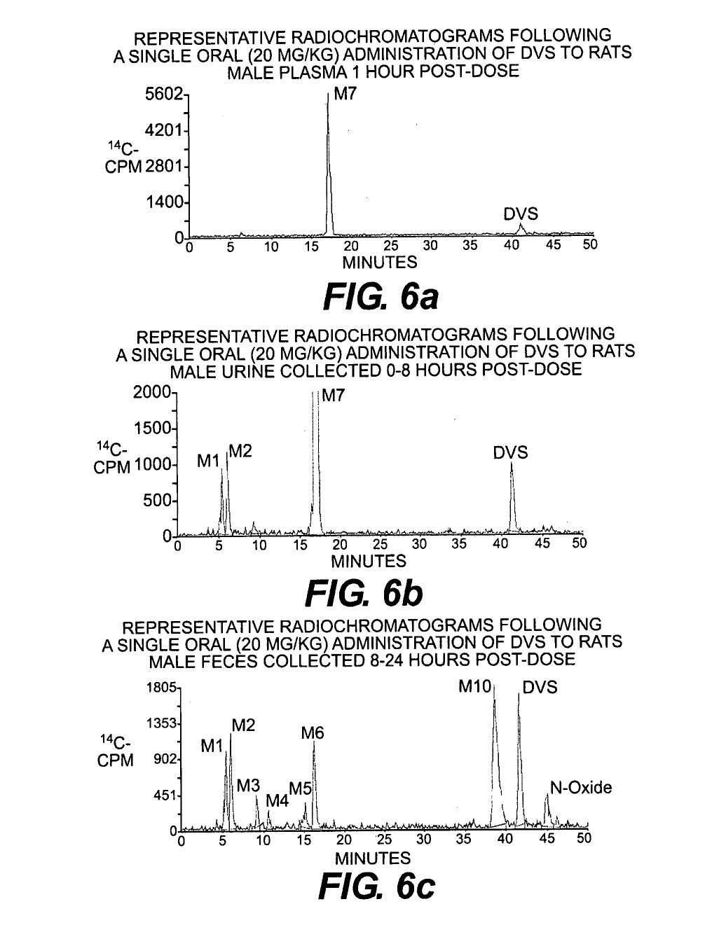

The average extraction efficiency of radioactivity from plasma was 98.7±13.0% (data not shown). A representative radiochromatogram of rat plasma collected from male rats 1 hour post-dose is shown in FIG. 6(A). At 1 and 4 hours post-dose, DV glucuronide (listed as M7 in Table 4) was the predominant peak detected by radiochromatography. At 1 and 4 hours post-dose, in male rats, 88.7 and 93.6% of the radioactivity in plasma was associated with the DV glucuronide peak, respectively. In female rats, DV glucuronide accounted for 86.6% of the radioactivity in plasma at 1 hour post-dose. The 8 and 24 hour samples did not have sufficient radioactivity to be analyzed by radiochromatography. The only other major radiochromatographic peak in the plasma samples was unchanged DVS, accounting for between 2.6 and 10% of the radioactivity in plasma, when it was detected. Other minor metabolites detected in some of the plasma samples included metabolites hydroxylated on the cyclohexane ring (M1-M6, hydroxy DV compounds). Individually, M1-M6 accounted for less than 2% of the radioactivity in plasma at each time point.

Additional minor metabolites, not present in the radiochromatogram, were detected and characterized by LC/MS in rat plasma (Table 5). These metabolites included N-oxide DV, N,O-didesmethylvenlafaxine (M10), N,O-didesmethylvenlafaxine O-glucuronide (M13).

TABLE 5
METABOLITES OF DVS OBSERVED BY LC/MS IN RAT PLASMA, URINE AND FECES

| | Retention | | | | |
|---|---|---|---|---|---|
| Metabolite | Time (min) | [M + H]⁺ | Site of Metabolism | Metabolite Name | Matrix^{a} |
| M1 | 4.1 | 280 | Cyclohexane ring | Hydroxy DV | P, U, F |
| M2 | 4.5 | 280 | Cyclohexane ring | Hydroxy DV | P, U, F |
| M3 | 7.4 | 280 | Cyclohexane ring | Hydroxy DV | P, U, F |
| M4 | 8.9 | 280 | Cyclohexane ring | Hydroxy DV | P, U, F |
| M5 | 13.0 | 280 | Cyclohexane ring | Hydroxy DV | P, U, F |
| M13 | 14.1 | 426 | Dimethylamino group and | N-Desmethyl DV O-Glucuronide | P, U |
| | | | Phenol —OH group | | |
| M7 | 14.4 | 440 | Phenol —OH group | DV O-Glucuronide | P, U, F |
| M6 | 14.6 | 280 | Cyclohexane ring | Hydroxy DV | P, U, F |
| M10 | 33.7 | 250 | Dimethylamino group | N,O-didesmethylvenlafaxine | P, U, F |
| | 34.9 | 264 | None | DV | P, U, F |
| | 36.7 | 280 | Dimethylamino group | N-Oxide DV | P, U, F^{b} |

^{a}P: plasma; U: urine; F: feces. Bold face type indicates that the metabolite was also detected by radiochromatography.
^{b}N-oxide was not detected in fecal samples by LC/MS, but was observed using radiochromatography.

### Urinary Metabolite Profiles

Urine was the predominant route of excretion, with greater than 50% of the radioactive dose recovered in urine samples within the first 8 hours post-dose and 85% recovered within 24 hours post-dose. The radioactivity concentrations detected in urine are shown in Table 6, as are the percent distribution of the radioactivity following radiochromatographic analysis. A representative radiochromatogram of rat urine collected 0-8 hours post-dose is shown in FIG. 6(B). The predominant radioactive peak detected in all samples analyzed was DV glucuronide (M7), which accounted for approximately 75% of the radioactive peaks detected in all urine samples at each time point. Unchanged [¹⁴C]DVS accounted for between 9 and 20% of the radioactivity detected in urine. Small amounts of two hydroxyl-DV compounds were detected in urine by radiochromatography. One of these with M2 being the most abundant of these metabolites, accounting for up to 7.5% of the radioactivity in urine.

TABLE 6
URINE CONCENTRATIONS AND PERCENT DISTRIBUTION OF THE
RADIOACTIVITY FOLLOWING A SINGLE ORAL (20 MG/KG) ADMINISTRATION
OF [¹⁴C]DVS TO RATS

| Sampling | | | | | Compounds Detected by | | | |
|---|---|---|---|---|---|---|---|---|
| Time | Radioactivity as % of Dose | | | | Radiochromatography (Mean ± S.D., n = 3)^{a} | | | |
| (hr/sex) | Individuals | | | Mean ± S.D. | M1 | M2 | M7 | DVS |
| 0-8/M | 60.1 | 50.7 | 66.5 | 59.1 ± 7.9 | 5.1 ± 0.9 | 7.5 ± 0.9 | 76.5 ± 1.9 | 10.9 ± 2.1 |
| 0-8/F | 53.8 | 42.7 | 67.1 | 54.5 ± 12 | 0.9 ± 0.2 | 5.1 ± 0.4 | 74.0 ± 3.3 | 20.0 ± 3.5 |
| 8-24/M | 24.6 | 32.3 | 19.7 | 25.5 ± 6.4 | 6.3 ± 0.5 | 7.4 ± 1.2 | 77.2 ± 1.2 | 9.1 ± 1.1 |

^{a}Values are expressed as percent of total peaks detected by radiochromatography.

Additional minor metabolites, not present in the radiochromatogram, were detected and characterized by LC/MS in urine (Table 5). These metabolites included M3, M4, M5, M6, N-oxide DV, N,O-didesmethylvenlafaxine (M10), N,O-didesmethylvenlafaxine O-glucuronide (M13).

### Fecal Metabolite Profiles

The efficiency of extraction of radioactivity from the 8-24 hour fecal samples prior to radiochromatographic analysis was 74.8±1.9% (data not shown). Only a small percentage of the radioactive dose (approximately 10%) was excreted in feces within 24 hours of dosing. Less than 0.1% of the radioactive dose was excreted in 0-8 hour fecal samples. The percent recovery in feces and the distribution of the radioactivity following radiochromatography analysis from individual rats are shown in Table 7. A representative radiochromatogram of extracted rat feces collected 8-24 hours post-dose is shown in FIG. 6(C). The most abundant peak detected by radiochromatography was N,O-didesmethylvenlafaxine (M10), accounting for 34% of the radioactivity in feces. Approximately 21% of the radioactivity in feces was unchanged DVS. N-oxide DV accounted for 7% of the radioactivity in feces. Combined, the hydroxylated metabolites M1-M6 accounted for approximately 38.6% of the radioactivity in feces, with the individual metabolites ranging from 1.7 to 12.2% of the radioactivity in feces. A small amount of O-desmethylvenlafaxine O-glucuronide (M7) was observed in feces only by LC/MS.

TABLE 7
CONCENTRATION AND PERCENT DISTRIBUTION OF THE
RADIOACTIVITY IN FECES COLLECTED 8-24 HOURS POST-DOSE
FOLLOWING A SINGLE ORAL (20 MG/KG) ADMINISTRATION OF [¹⁴C]DVS TO
RATS

| | Radioactivity | Compounds Detected by Radiochromatography^{a} | | | | | | | | |
|---|---|---|---|---|---|---|---|---|---|---|
| Rat Number | as % of Dose | M1 | M2 | M3 | M4 | M5 | M6 | M10 | DVS | N-Oxide |
| 010M | 8.5^{b} | 8.0 | 10.4 | 3.1 | 1.8 | 1.7 | 11.0 | 33.6 | 22.0 | 8.5 |
| 011M | 10.8 | 9.5 | 9.7 | 3.2 | 1.8 | 3.6 | 12.2 | 34.5 | 19.6 | 6.0 |
| 012M | 9.5 | 8.8 | 9.9 | 3.9 | 3.9 | 2.1 | 11.4 | 32.6 | 20.8 | 6.7 |
| Mean ± S.D. | 9.6 ± 1.1 | 8.8 ± 0.8 | 10.0 ± 0.4 | 3.4 ± 0.5 | 2.5 ± 1.2 | 2.5 ± 1.0 | 11.5 ± 0.6 | 33.6 ± 0.9 | 20.8 ± 1.2 | 7.1 ± 1.3 |

^{a}Values are expressed as percent of total peaks detected by radiochromatography.
^{b}Fecal samples collected from male and female rats from 0-8 hours post-dose contained 0.017 and 0.025% of the radioactive dose, respectively, and were not analyzed by radiochromatography.

### Metabolite Characterization by Liquid Chromatography/Mass Spectrometry

Mass spectra were obtained by LC/MS and LC/MS/MS analysis for DVS and its metabolites in rat plasma, urine, and feces. Structural characterization of the DVS metabolites in rat is summarized in Table 5. LC/MS data indicated metabolism of DVS to a glucuronide (M7), N,O-didesmethylvenlafaxine (M10), and hydroxylation products (M1-M6). The mass spectral characterization of these metabolites, DVS, N-oxide DV, and a minor metabolite (M13) is discussed below.

### DVS

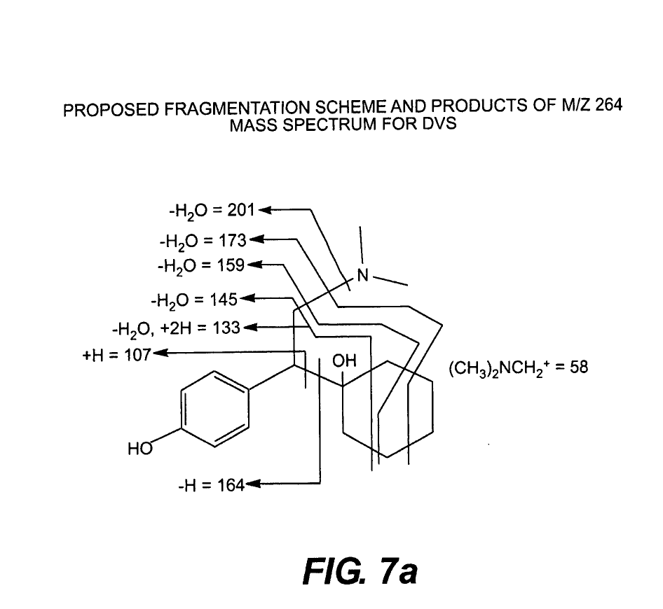

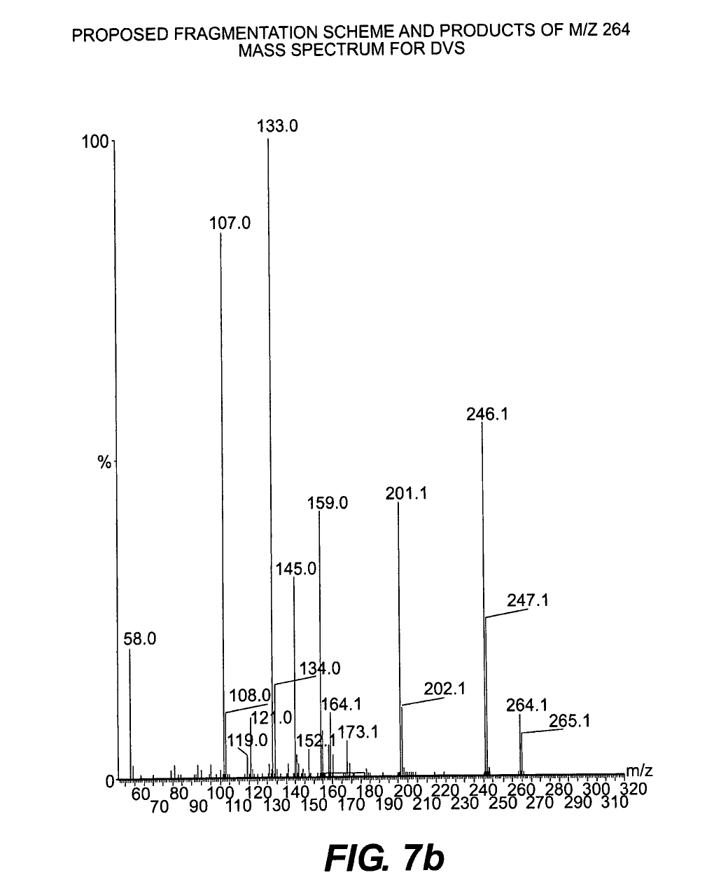

The mass spectral characteristics of a DVS standard were examined for comparison with metabolites. In the LC/MS spectrum of DVS, a protonated molecular ion, [M+H]⁺ was observed at m/z 264. FIG. 7 shows the products of m/z 264 mass spectrum of DVS, obtained from collision induced dissociation (CID), and the proposed fragmentation scheme. Loss of H₂O from the molecular ion yielded the product ion at m/z 246. Further loss of the dimethylamino group yielded the product ion at m/z 201. Loss of the cyclohexanol group from DVS was represented by the product ion at m/z 164. The product ion at m/z 58 was due to (CH₃)₂NCH₂⁺. In addition, the product ions at m/z 107, 133, 145, 159 and 173 corresponded to the methyl, propyl, butyl, pentyl and hexyl-phenolic portions, respectively, of the DVS molecule. Therefore, these ions could be used to detect sites of metabolism localized to the dimethylamino, hydroxybenzyl and cyclohexanol groups.

### Metabolites M1, M2, M3, M4, M5, and M6 (Hydroxy DV Compounds)

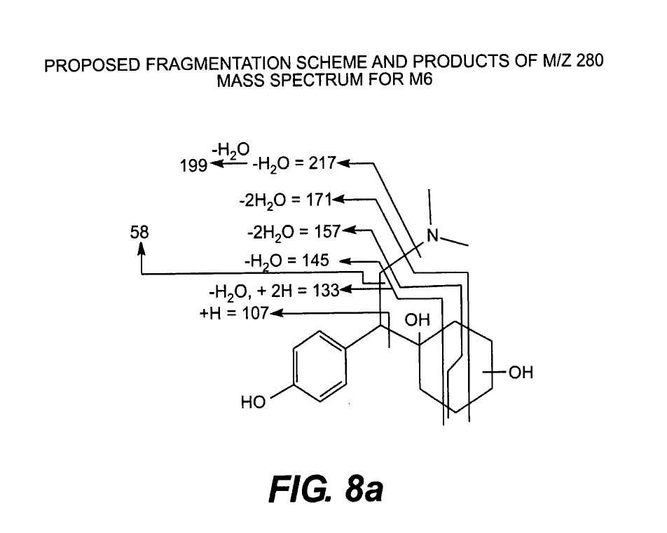

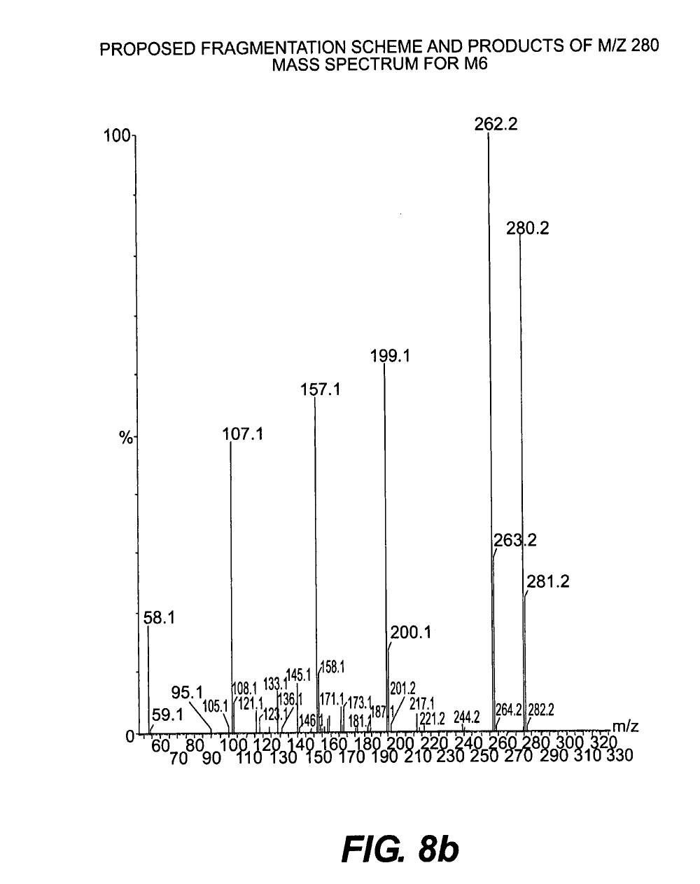

Metabolites M1 to M6 produced a [M+H]⁺ at m/z 280, which was 16 Da larger than DVS and suggested hydroxylation or N-oxidation. FIG. 8 shows the products of m/z 280 spectrum for M6. Mass spectral data for metabolites M1 to M6 were similar. Loss of H₂O from the molecular ion yielded the product ion at m/z 262. The product ions at m/z 58, 107 and 217 for the metabolites versus at m/z 58, 107 and 201 for DVS indicated the cyclohexane ring as the site of metabolism. Therefore, metabolites M1 through M6 were proposed to be hydroxy DVS metabolites with the cyclohexane ring as the site of oxidation.

Metabolite M7 (O-desmethylvenlafaxine O-glucuronide, DV glucuronide)

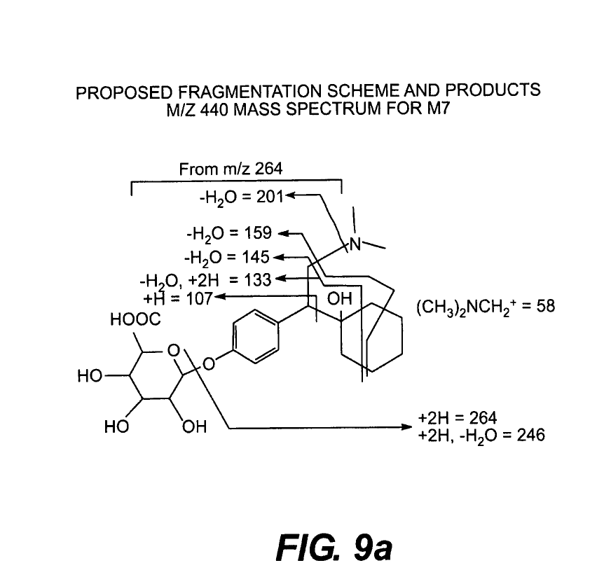

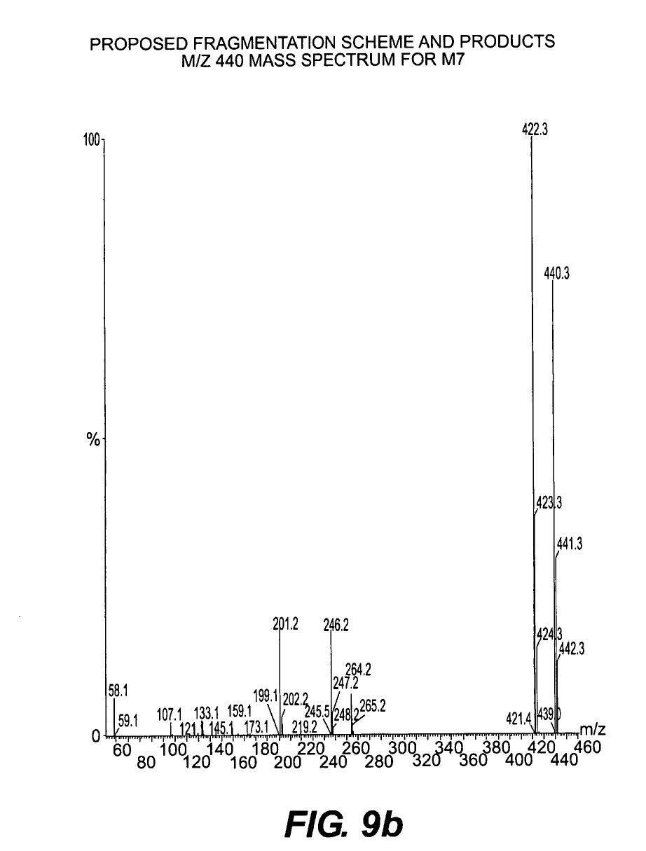

The [M+H]⁺ for this metabolite was observed at m/z 440, which indicated a molecular weight of 439. FIG. 9 shows the products of m/z 440 spectrum for M7. The loss of 176 Da from the molecular ion yielded the ion at m/z 280, which indicated that this metabolite was a glucuronide. Product ions at m/z 246, 201, 159, 145, 133, 107 and 58 were also observed for DVS. The mass spectral data did not indicate the site of conjugation. However, DVS undergoes the same loss of H₂O to generate a [MH-H₂O]⁺ at m/z 246 (FIG. 7). These losses of H₂O had occurred from the cyclohexanol group. This indicates that phenol, rather than the cyclohexanol, is the site of glucuronidation. Additionally, the phenol group was the more metabolically likely site of conjugation. Therefore, M7 was identified as an O-glucuronide of DVS with the phenol group as the site of conjugation.

### Metabolite M10 (N,O-didesmethylvenlafaxine)

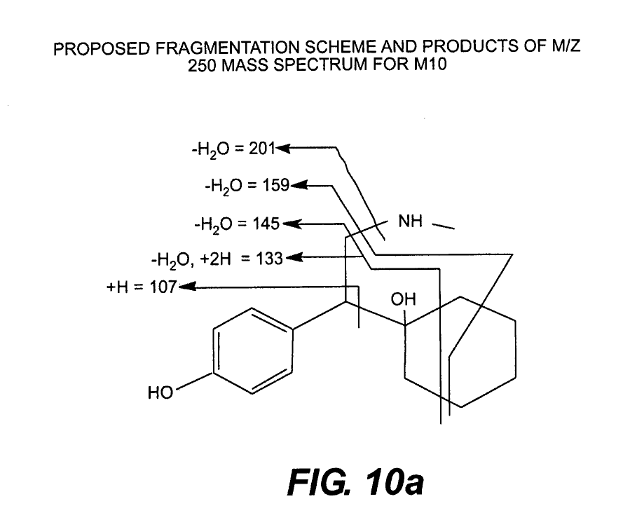

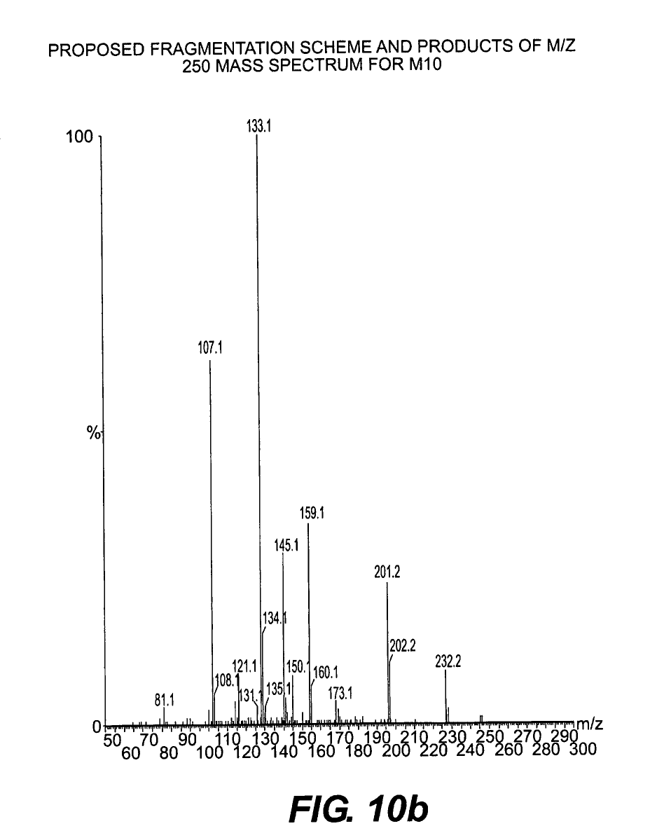

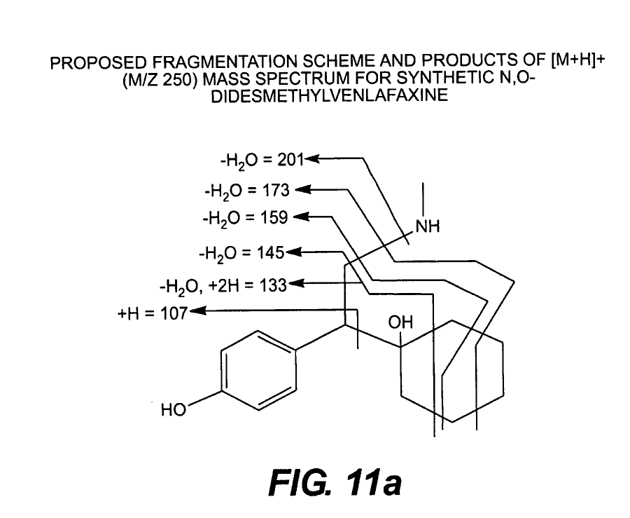

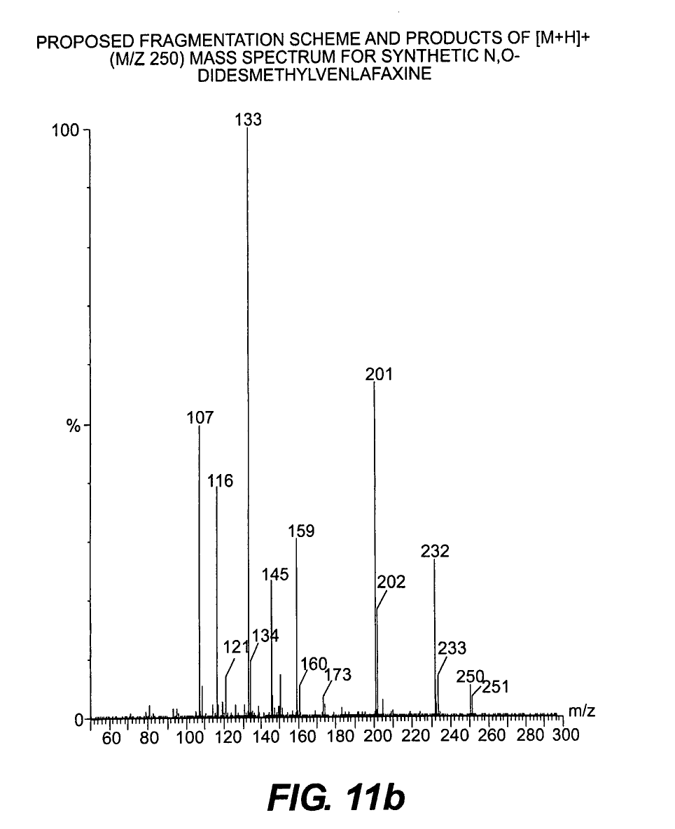

The [M+H]⁺ for M1 was observed at m/z 250. FIG. 10 shows the products of m/z 250 spectrum for M10. Loss of H₂O from the molecular ion at m/z 250 yielded the product ion at m/z 232. Subsequent loss of methylamine from m/z 232 generated the diagnostic product ion at m/z 201. This, and the lack of a product ion at m/z 58, indicated that the dimethylamino group of DVS had been converted to a methylamino group by N-demethylation. The products of m/z 250 mass spectrum for M10 matched the products of m/z 250 mass spectrum for synthetic N,O-didesmethylvenlafaxine. FIG. 11 shows the products of m/z 250 mass spectrum for synthetic N,O-didesmethylvenlafaxine. Therefore, M10 was identified as N,O-didesmethylvenlafaxine.

### Metabolite M13 (N,O-didesmethylvenifaxine O-glucuronide)

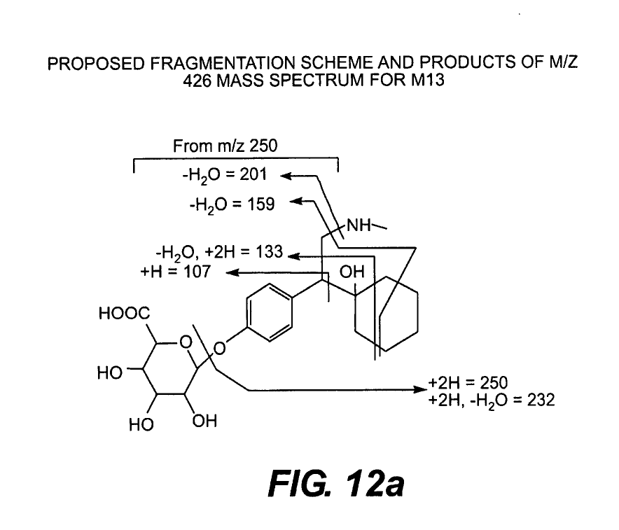

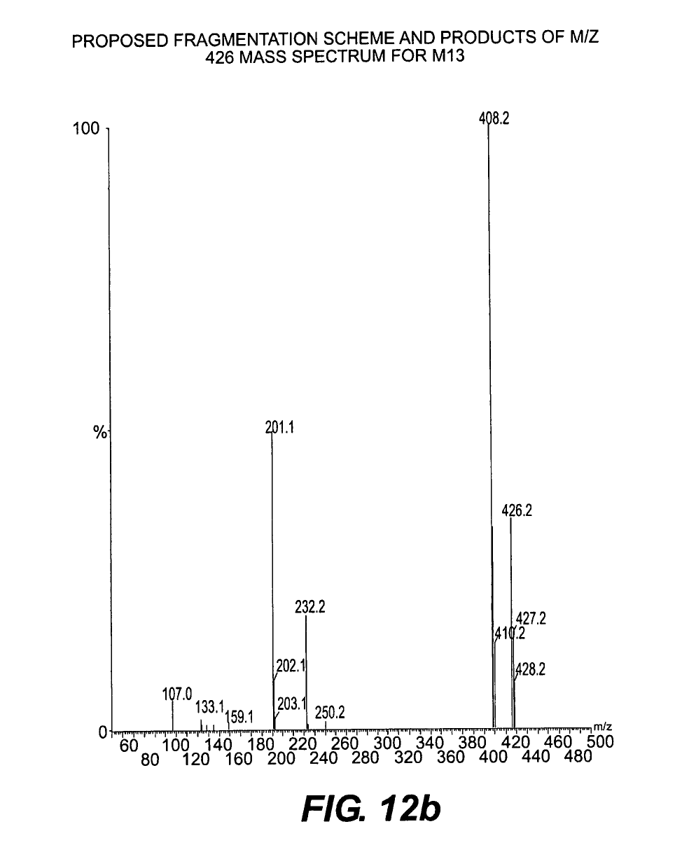

The [M+H]⁺ for this metabolite was observed at m/z 426, which indicated a molecular weight of 425. FIG. 12 shows the product ion spectrum of M13. The loss of 176 Da from m/z 426 yielded the ion at m/z 250. Loss of H₂O from the cyclohexanol moiety yielded the base peak at m/z 408. The loss of 176 Da from the ion at m/z 408 yielded the diagnostic product ion of M10 at m/z 232. Subsequent loss of methylamine from m/z 232 generated the product ion at m/z 201. Therefore, M13 was proposed to be the N,O-didesmethylvenlafaxine O-glucuronide with the phenol group as the site of glucuronidation.

### N-Oxide DV

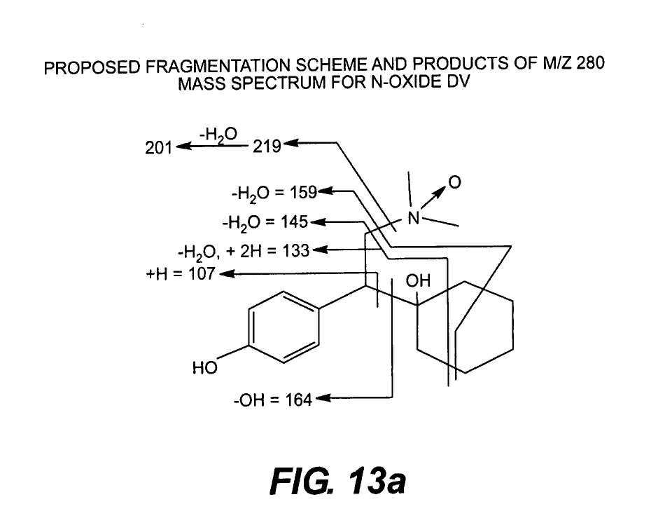

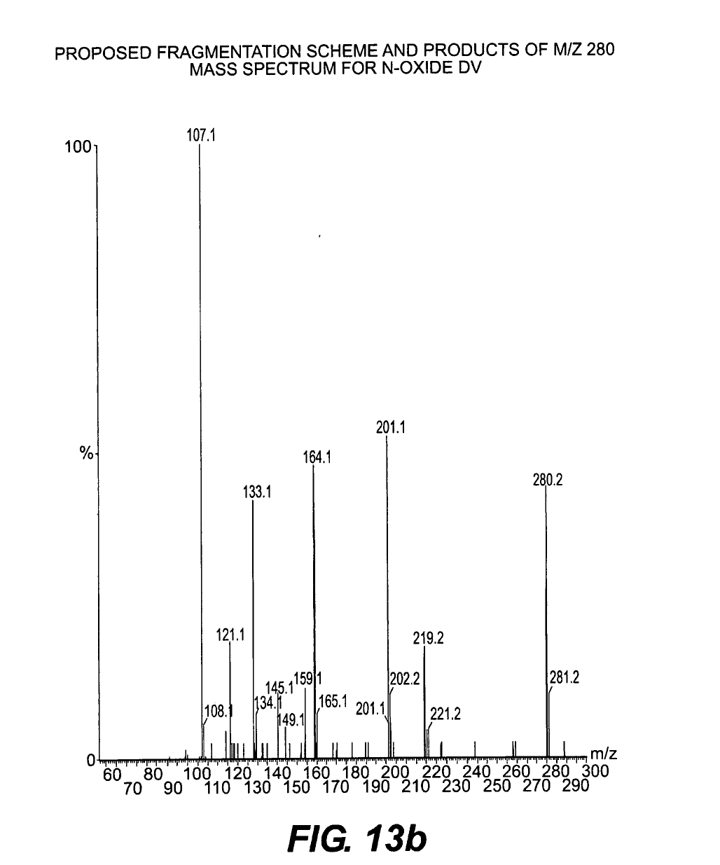

The [M+H]⁺ for this DVS related component was observed at m/z 280, which indicated hydroxylation or N-oxidation. FIG. 13 shows the products of m/z 280 mass spectrum for this DVS related compound. Loss of 61 Da from [M+H]⁺ ion yielded the product ion at m/z 219. This corresponded to loss of dimethylhydroxyamine consistent with an N-oxide. Therefore, this metabolite was identified as the N-oxide of DVS.

### Example 2

### Metabolism of [¹⁴C]O-Desmethylvenlafaxine in Beagle Dogs Following a Single Oral Administration

(2 or 3)-Hydroxy DV compounds, hydroxy DV glucuronides, N-oxide DV compounds, as well as other compounds, and a benzyl hydroxy compound were detected in the metabolic profiles for [¹⁴C]DVS in urine, feces, and plasma following a single oral gavage dose in male beagle dogs as described below.

### Materials and Methods

Radiolabeled [¹⁴C]DVS (Batch #CFQ13003, [cyclohexyl-1-¹⁴C]DVS) was supplied by Amersham Biosciences (Buckinghamshire, UK). Unlabeled DVS (Batch RB1636; free base 65.2%) was received from Wyeth Research, Rouses Point, N.Y. The average molecular weight of DVS is 381.5, with the free base, O-desmethylvenlafaxine, accounting for 69.0% by weight. The specific activity of [¹⁴C]DVS (bulk drug) was 144 μCi/mg (209 μCi/mg for the free base) and the radiopurity of the free base was 99.3%, as determined by HPLC using radiometric detection.

Water for preparation of the oral dosing solution was obtained from EM Science (Gibbstown, N.J.). Methylcellulose and polysorbate 80 were received from Sigma Chemical Co. (St. Louis, Mo.) and Mallinckrodt Baker (Phillipsburg, N.J.), respectively. The liquid scintillation cocktail used in counting the radioactivity in urine and plasma samples, fecal homogenate extracts and the dosing solution aliquots was Ultima Gold™ (Perkin Elmer, Wellesley, Mass.). A model 307 Tri-Carb Sample Oxidizer, equipped with an Oximate-80 Robotic Automatic Sampler (Perkin Elmer), was used for combustion of blood and fecal samples. PermaFluor® E⁺ liquid scintillation cocktail (Perkin Elmer), Carbosorb® E (Perkin Elmer) carbon dioxide absorber and HPLC grade water were used to trap radioactive carbon dioxide generated by combustion of the samples in the oxidizer. Fecal homogenates and blood samples were transferred to combusto-cones and cover pads (Perkin Elmer) for combustion.

### Animals

Male beagle dogs (n=4), weighing between 14.4 and 16.2 kg at the time of dosing (from an in-house colony), were used. For ease of reporting, the animals were designated numbers 5 through 8. Dose preparation, animal dosing and sample collection were performed at Wyeth Research, Pearl River, N.Y.

### Dose Preparation, Dosing and Analysis

The oral dosing solution was prepared by suspending 19.0 mg of [¹⁴C]DVS and 4168.3 mg of unlabeled DVS in 270 mL of vehicle (0.25% polysorbate 80, 0.5% methylcellulose in water). The radiochemical purity, specific activity and concentration of [¹⁴C]DVS (bulk drug and dosing solution) were determined using HPLC with radiometric detection. Duplicate aliquots of the dosing solution were taken pre-, mid- and post-dose for the determination of specific activity and radioactivity concentrations of the dosing solution.

The target dose for each animal was 30 mg/kg (free base; 10 mg/mL, 3 mL/kg, 30 μCi/kg) [¹⁴C]DVS via oral gavage. The target dose was selected because it has been used in previous pharmacokinetic studies. Additionally, this dose, administered subcutaneously, significantly increased the norepinephrine levels in the brains of male Sprague Dawley rats.

### Blood Collection and Analysis

Whole blood (approximately 10 mL), collected into heparinized tubes at 1, 4, 8, and 24 hours post-dose (N=4 for each time point), was analyzed. One mL of blood was transferred to a fresh tube to be used for determination of radioactivity concentrations. Plasma was obtained by centrifugation at 4° C. within two hours of blood collection. Plasma and whole blood samples were shipped on dry ice to Wyeth Research, Biotransformation Division (Collegeville, Pa.) for analysis. Triplicate aliquots of whole blood (200 μL) were placed into combusto-cones and allowed to air dry. These samples were then oxidized and radioactivity content determined. Triplicate aliquots (100 μL) of the plasma samples were analyzed for radioactivity content. The remaining plasma was stored at −70° C. until metabolite analysis.

For each dog, urine and feces were collected separately, with urine collected on dry ice and feces collected at room temperature. Collections were from 0-8 and 8-24 hours for urine and 0-24 hours for feces. Urine and fecal samples were shipped on dry ice to Wyeth Research, Biotransformation Division (Collegeville, Pa.) for analysis. Fecal samples were homogenized in approximately five volumes (v/w) of water. Aliquots of approximately 0.2 grams of the homogenate were placed into combusto-cones, weighed and allowed to air dry. These samples were then oxidized and radioactivity content determined. The remaining urine samples and fecal homogenates were stored at −70° C. until metabolite analysis. Blood samples and fecal homogenates were oxidized in a Model 307 Tri-Carb sample oxidizer, using Carbosorb® E (6 mL) as trapping agent and PermaFluor® E⁺ (10 mL) as scintillant. The background reading (average of control blood or fecal samples) was subtracted from each sample reading. Aliquots of urine and plasma were analyzed directly following the addition of 10 mL of Ultima Gold™ scintillation fluid.

All radioactivity determinations were made using a Tri-Carb Model 3100TR liquid scintillation counter (Packard BioScience, Boston, Mass.) with an Ultima GOl™ or toluene standard curve. Counts per minute (CPM) were converted to disintegrations per minute (DPM) by use of external standards of known radioactivity. The quench of each standard was determined by the transformed spectral index of an external radioactive standard (tSIE). The lower limits of detection were defined as twice background.

### Plasma Metabolite Samples

Plasma samples collected at 1 and 4 hours post-dose were analyzed for metabolite profiles. Aliquots of 1 mL plasma were mixed with an equal volume of acetonitrile, placed on ice for at least 10 minutes, and then centrifuged at 14000 rpm in an Eppendorf Model 5415C centrifuge for 10 minutes. The supernatant fluid was transferred to a clean tube. The supernatant was analyzed for radioactivity. The supernatant was concentrated under a stream of nitrogen in a Turbo Vap (Zymark, Hopinkton, Mass.) to remove the acetonitrile. An aliquot of the aqueous residue was analyzed by HPLC for profiling. Selected samples were also analyzed by LC/MS to characterize the radioactive peaks.

The stability of [¹⁴C]DVS in dog plasma was determined. [¹⁴C]DVS (0.012 mg/mL, final concentration) was added to control dog plasma and incubated in a shaking water bath set to 37° C. Duplicate aliquots (1 mL) were removed at 0, 1, 4, 8, and 24 hours. Samples were extracted as described above and radiopurity assayed by HPLC analysis.

### Urine Metabolite Samples

Urine samples collected between 8 and 24 hours post-dose were analyzed for metabolite profiles. Aliquots of urine were centrifuged at 14000 rpm in an Eppendorf Model 5415C centrifuge for 10 minutes. The supernatant was transferred to a fresh tube and analyzed for radioactivity content and by HPLC for metabolite profiling. Selected samples were also analyzed by LC/MS to characterize the radioactive peaks.

The stability of [¹⁴C]DVS in dog urine was determined. [¹⁴C]DVS (0.025 mg/mL, final concentration) was added to control dog urine and incubated in a shaking water bath set to 37° C. Aliquots (1 mL) were removed at 0, 1, 4, 8 and 24 hours. Samples were extracted as described above and radiopurity assayed by HPLC analysis.

### Fecal Metabolite Samples

Fecal homogenates collected up to 24 hours post-dose were analyzed for metabolite profiles. Aliquots of approximately 2 grams of fecal homogenate were transferred to a fresh tube, an equal volume of acetonitrile (v/w) was added, and the tube vortexed. Samples were then centrifuged at 14000 rpm in an Eppendorf Model 5415C centrifuge for 10 minutes. The supernatant was transferred to a clean tube. The residue was re-suspended with 1 mL acetonitrile and centrifuged as described above. The resulting supernatant was combined with the original supernatant and analyzed for radioactivity. The supernatants were then concentrated under a stream of nitrogen in a Turbo Vap to remove the acetonitrile. An aliquot of the aqueous residue was analyzed by HPLC for profiling. Selected samples were also analyzed by LC/MS to characterize the radioactive peaks.

### Sample Analysis

Chromatographic analyses were performed with a Waters Alliance model 2690 HPLC system (Waters Corp., Milford, Mass.). It was equipped with a built-in autosampler and was in-line with a model 2487 tunable UV detector, set to monitor 225 nm, and a FloOne β Model 515 radioactivity flow detector (Perkin Elmer) with a 250 μL LQTR flow cell. The flow rate of Ultima Flow M scintillation fluid was 3 mL/min, providing a mixing ratio of scintillation cocktail to mobile phase of 3:1. Separation of the metabolite peaks was accomplished on a Phenomenex Luna C18(2) column, 250×4.6 mm, 5 micron (Phenomenex, Torrance, Calif.), using a linear gradient of two mobile phases, A and B. Mobile phase A was 10 mM ammonium acetate, pH 5.5, and mobile phase B was acetonitrile. The flow rate was 1 mL/min. The mobile phase was delivered as shown in Table 8.

TABLE 8
CHROMATOGRAPHIC MOBILE
PHASE DELIVERY CONDITIONS.

| Time (min) | A (%) | B (%) |
|---|---|---|
| 0 | 95 | 5 |
| 30 | 85 | 15 |
| 40 | 85 | 15 |
| 41 | 10 | 90 |
| 46 | 10 | 90 |
| 47 | 95 | 5 |

An Agilent Model 1100 HPLC system (Agilent Technologies, Wilmington, Del.) including an autosampler and diode array UV detector was used for LC/MS analysis. The UV detector was set to monitor 200 to 400 nm. Separations were accomplished on a 5 micron Phenomenex Luna C18(2) column, 150×2 mm (Phenomenex). The column temperature was 25° C. The mobile phases and gradient program are listed in Table 2. For selected LC/MS analyses, radiochromatograms were acquired using a β-RAM model 3 radioactivity flow detector (IN/US Systems Inc., Tampa, Fla.) equipped with a solid scintillation flow cell.

The mass spectrometers used for metabolite characterization were a Micromass Q-TOF-2 quadrupole time-of-flight hybrid mass spectrometer (Micromass, Inc., Beverly, Mass.) and a Finnigan LCQ Deca ion trap mass spectrometer (ThermoFinnigan, San Jose, Calif.). The mass spectrometer was equipped with an electrospray ionization (ESI) interface and operated in the positive ionization mode. Settings for the mass spectrometers are listed in Table 9 and Table 10.

TABLE 9
MICROMASS Q-TOF-2 MASS
SPECTROMETER SETTINGS

| | |
|---|---|
| Capillary Voltage | 3.2 kV |
| Cone | 28 V |
| Source Block | 80° C. |
| Temperature | |
| Desolvation Temperature | 200° C. |
| Desolvation Gas Flow | 350 L/hr |
| Cone Gas Flow | 75 L/hr |
| CID Gas Inlet Pressure | 13-14 psig |

TABLE 10
FINNIGAN LCQ ION TRAP MASS
SPECTROMETER SETTINGS

| Nebulizer gas | 80 arb. uinits |
|---|---|
| Auxiliary gas | 10 arb. units |
| Spray voltage | 5.0 KV |
| Heated capillary temp. | 300° C. |
| Full scan AGC setting | 5 × 10⁷ |
| Relative collision energy | 35% |

To confirm the site of glucuronidation of DVS, incubations were performed using dog liver microsomes. These incubations compared the glucuronidation of DVS to venlafaxine. Briefly, venlafaxine or DVS (100 μM) was incubated with dog liver microsomes (1 mg/mL) and MgCl₂ (10 mM) in 0.1 M sodium/potassium phosphate buffer. Samples were pre-incubated for 2 minutes in a shaking water bath set to 37° C. Reactions were initiated by the addition of UDPGA (final concentration 1 mM). An additional set of incubations was performed for venlafaxine with UDPGA and an NADPH generating system. The total incubation volume was 500 μL and the length of incubation was 30 minutes. Reactions were stopped by the addition of 500 μL of acetonitrile and processed as described above. Samples were analyzed by LC/MS.

FloOne analytical software (version 3.65, Packard BioScience) was utilized to integrate the radioactive peaks. The computer program Microsoft Excele 97 was used to calculate means and standard deviations. MassLynx Software (version 3.5) was used to analyze the LC/MS data.

### Results

The radiochemical purity and specific activity of [¹⁴C]DVS (bulk compound), determined by HPLC with radiometric detection, were 99.3% and 209 μCi/mg (free base), respectively. The concentration, radiopurity and specific activity of [¹⁴C]O-desmethylvenlafaxine in the dosing solution were 10.3 mg/mL, 98.3% and 1.03 μCi/mg, respectively. Pre-, mid- and post-dose aliquots of the dosing solution had similar concentrations and purities (data not shown). The mean administered dose of [¹⁴C]DVS was 31.0±0.18 mg/kg (free base).

[¹⁴C]DVS was stable at 37° C. for up to 24 hours in control dog urine and control dog plasma. No significant degradation products were detected by radiochromatography at any of the time points up to and including 24 hours. Oxidation efficiency was determined by oxidation of ¹⁴C-Spec-Chec (Perkin Elmer), a standard of known radioactivity, and determined to be 99.1%. The concentrations of radioactivity in blood and plasma, and the blood to plasma partitioning for each time point are shown in Table 11. The mean plasma concentrations of total radioactivity in male dogs were 13.3, 16.9, 7.43, and 0.81 μg equivalents/mL at 1, 4, 8, and 24 hour post-dose, respectively. At each time point the blood to plasma ratio for radioactivity ranged between 0.51 and 0.64.

TABLE 11
WHOLE BLOOD AND PLASMA RADIOACTIVITY CONCENTRATIONS
AND PARTITIONING OF THE RADIOACTIVITY FOLLOWING A SINGLE ORAL
(30 MG/KG) ADMINISTRATION OF [¹⁴C]DVS TO DOGS

| | | | | | | | | | | | Blood to |
|---|---|---|---|---|---|---|---|---|---|---|---|
| | Radioactivity in Whole Blood | | | | | Radioactivity in Plasma | | | | | Plasma |
| Sampling | (μg equivalents/mL) | | | | | (μg equivalents/mL) | | | | | Ratio |
| Time | Individual Dogs | | | | Mean ± S.D. | Individual Dogs | | | | Mean ± S.D. | Mean ± S.D. |
| 1 hr | 8.56 | 8.61 | 6.40 | 9.91 | 8.37 ± 1.45 | 14.6 | 11.5 | 9.56 | 17.5 | 13.3 ± 3.5 | 0.64 ± 0.09 |
| 4 hr | 8.16 | 8.03 | 8.82 | 9.30 | 8.58 ± 0.59 | 17.0 | 16.8 | 16.5 | 17.1 | 16.9 ± 0.3 | 0.51 ± 0.04 |
| 8 hr | 3.30 | 3.79 | 5.27 | 2.98 | 3.84 ± 1.01 | 4.51 | 9.12 | 10.2 | 5.87 | 7.43 ± 2.68 | 0.54 ± 0.13 |
| 24 hr | 0.38 | 0.47 | 0.75 | 0.31 | 0.48 ± 0.20 | 0.66 | 0.86 | 1.14 | 0.56 | 0.81 ± 0.25 | 0.58 ± 0.05 |

### Plasma Metabolite Profiles

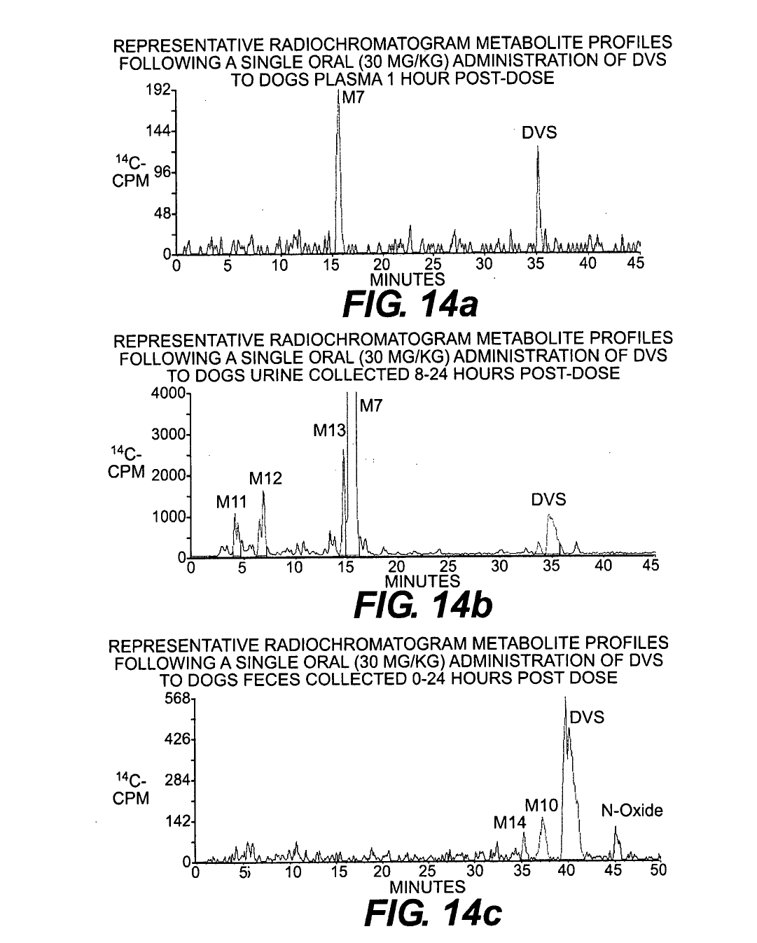

The average extraction efficiency of radioactivity from plasma was 87.6±10.1% (data not shown). A representative radiochromatogram of dog plasma collected 1 hour post-dose is shown in FIG. 14(A). DV glucuronide (M7) was the predominant peak detected. At 1 and 4 hours post-dose 77.5 and 96.4% of the radioactivity detected in plasma was associated with the M7 peak. The 8 and 24 hour samples did not have sufficient radioactivity for radiochromatographic analysis. The only other radioactive component detected in plasma was unchanged DVS. Nine additional minor metabolites were characterized by LC/MS in dog plasma (Table 12). These metabolites included six metabolites hydroxylated on the cyclohexanol ring (M1-M6, hydroxy DV compounds), N,O-didesmethylvenlafaxine (M10), N,O-didesmethylvenlafaxine O-glucuronide (M13), and N-oxide DV.

TABLE 12
METABOLITES OF DVS OBSERVED BY LC/MS IN DOG PLASMA, URINE AND FECES

| | Retention | | | | |
|---|---|---|---|---|---|
| Metabolite | Time (min)^{a} | [M + H]⁺ | Site of Metabolism | Name of Metabolite | Matrix^{b} |
| M11a, b, c | 3.5, 3.6, 3.8 | 456 | Cyclohexane ring and | Hydroxy DV O-Glucuronide | U |
| | | | Phenol —OH group | | |
| M12 | 5.3 | 456 | Cyclohexane ring and | Hydroxy DV O-Glucuronide | U |
| | | | Phenol —OH group | | |
| M1 | 5.4 | 280 | Cyclohexane ring | Hydroxy DV | P, U, F |
| M2 | 6.2 | 280 | Cyclohexane ring | Hydroxy DV | P, U, F |
| M3 | 9.7 | 280 | Cyclohexane ring | Hydroxy DV | P, U, F |
| M4 | 10.9 | 280 | Cyclohexane ring | Hydroxy DV | P, U, F |
| M13 | 13.9 | 426 | Dimethylamino group | N-Desmethyl DV O-Glucuronide | P, U |
| | | | and Phenol —OH | | |
| | | | group | | |
| M7 | 14.8 | 440 | Phenol —OH group | DV O-Glucuronide | P, U, F |
| M5 | 15.7 | 280 | Cyclohexane ring | Hydroxy DV | P, U, F |
| M6 | 16.4 | 280 | Cyclohexane ring | Hydroxy DV | P, U, F |
| M9 | 30.0 | 280 | Benzyl group | Hydroxy DV | U, F |
| M14 | 32.2 | 236 | Dimethylamino group | N-Didesmethyl DV | U, F |
| M10 | 32.9 | 250 | Dimethylamino group | N-Desmethyl DV | P, U, F |
| | 33.7 | 264 | None | DV | P, U, F |
| | 36.8 | 280 | Dimethylamino group | DV N-Oxide | P, U, F |

^{a}LC/MS retention time taken from data file GU_070202_0003.
^{b}P: plasma; U: urine; F: feces. Bold face type indicates that the metabolite was also detected by radiochromatography.

### Urinary Metabolite Profiles

Urine was the predominant route of excretion, with an average of 75% of the radioactive dose recovered in urine samples within 24 hours post-dose. The radioactivity concentrations detected in urine are shown in Table 13, as are the percent distribution of the radioactivity following radiochromatography. A representative radiochromatogram of dog urine collected 8-24 hours post-dose is shown in FIG. 14(B). The predominant radioactive peak detected in all urine samples analyzed was O-desmethylvenlafaxine O-glucuronide (M7, DV glucuronide), which accounted for approximately 85% of the radioactive peaks detected in urine. N,O-didesmethylvenlafaxine O-glucuronide (M13) accounted for approximately 4% of the drug-related peaks detected in urine. Unchanged [¹⁴C]DVS accounted for between 4 and 8% of the radioactivity detected in urine. Metabolites M11 and M12 (glucuronide conjugates of metabolites hydroxylated on the cyclohexane ring, “Hydrdoxy DV glucuronides”) accounted for averages of 2 and 4% of the radioactivity detected in urine, respectively. The M11 peak contained three co-eluting metabolites (M11a, M11b and M11c) that were each identified by LC/MS as glucuronide conjugates of metabolites hydroxylated on the cyclohexane ring.

TABLE 13
CONCENTRATION AND PERCENT DISTRIBUTION OF THE
RADIOACTIVITY IN URINE COLLECTED 8-24 HOURS POST-DOSE
FOLLOWING A SINGLE ORAL (30 MG/KG) ADMINISTRATION OF DVS TO
DOGS

| | Radioactivity | Compounds Detected by Radiochromatography^{a} | | | | |
|---|---|---|---|---|---|---|
| Dog Number | as % of Dose | M11 | M12 | M13 | M7 | DVS |
| 5 | 64.0 | 2.6 | 3.0 | 3.6 | 86.8 | 4.0 |
| 6 | 85.4 | 1.9 | 2.7 | 3.3 | 84.1 | 7.9 |
| 7 | 63.0 | 2.0 | 4.3 | 3.5 | 83.5 | 5.7 |
| 8 | 86.6 | 2.6 | 3.8 | 4.3 | 83.9 | 5.4 |
| Mean ± S.D. | 74.8 ± 13.0^{b} | 2.3 ± 0.4 | 3.5 ± 0.7 | 3.7 ± 0.4 | 84.6 ± 1.5 | 5.8 ± 1.6 |

^{a}Values are expressed as percent of total peaks detected by radiochromatography, mean of 2 analyses.
^{b}Values for % of dose include 0-8 and 8-24 hour time points, but 0-8 hour collection contained less than 0.1% of the dose.

Ten additional minor metabolites were characterized by LC/MS analysis of urine. These minor metabolites included M1-M6, a metabolite hydroxylated on the benzyl group (M9), N,O-didesmethylvenlafaxine (M10), N,N,O-tridesmethylvenlafaxine (M14), and N-oxide DV (Table 12).

### Fecal Metabolite Profiles

The efficiency of extraction of radioactivity from the 0-24 hour fecal samples prior to radiochromatography was 76.8±6.2% (data not shown). The percent recovery in feces and the distribution of the radioactivity following radiochromatography are shown in Table 14. Only a small percentage of the radioactive dose (approximately 3%) was excreted in feces within 24 hours of dosing. A representative radiochromatogram of extracted dog feces collected 0-24 hours post-dose is shown in FIG. 14(C). Four radioactive peaks were detected, with unchanged DVS being the predominant peak detected in each chromatogram, accounting for an average of 76% of the radioactivity in feces. The next most abundant radioactive peak was M10, accounting for approximately 12% of the radioactivity excreted in feces. N-oxide DV and N,N,O-tridesmethylvenlafaxine (M14) were also present in the radiochromatograms of the fecal extracts, accounting for approximately 7 and 5%, respectively.

TABLE 14
CONCENTRATION AND PERCENT DISTRIBUTION OF THE
RADIOACTIVITY IN FECES COLLECTED 0-24 HOURS POST DOSE
FOLLOWING A SINGLE ORAL (30 MG/KG) ADMINISTRATION OF
[¹⁴C]DVS TO DOGS

| Dog | Radioactivity | Compounds Detected by Radiochromatography^{b} | | | |
|---|---|---|---|---|---|
| Number | as % of Dose | M14 | M10 | DVS | N-Oxide |
| 5 | 3.3 | 0.0 | 11.2 | 88.8 | 0.0 |
| 6 | 4.4 | 4.4 | 9.8 | 78.6 | 7.3 |
| 7^{a} | 0.3 | 16.1 | 17.7 | 50.6 | 15.7 |
| 8 | 4.0 | 0.0 | 8.6 | 85.7 | 5.7 |
| Mean ± | 3.0 ± 1.9 | 5.1 ± 7.0 | 11.8 ± 4.9 | 75.9 ± 17.4 | 7.2 ± 6.1 |
| S.D. | | | | | |

^{a}At 24 hours post-dose there was no fecal sample for dog 7, so collection continued until 48 hours.
^{b}Values are expressed as percent of total peaks detected by radiochromatography, average of 2 analyses.

Eight additional minor metabolites, not detected in the radiochromatograms, were characterized by LC/MS analysis of the fecal extracts. These metabolites included M1-M6, M7, and M9 (Table 12).

### Metabolite Characterization by Liquid Chromatography/Mass Spectrometry

Mass spectra were obtained by LC/MS and LC/MS/MS analysis for DVS and its metabolites in dog plasma, urine, and feces. Structural characterization of the DVS metabolites in dog is summarized in Table 12. LC/MS data indicated metabolism of DVS to a glucuronide (M7), N-desmethyl DVS (M10), and mono-oxidation products. The mass spectral characterization of DVS and 14 metabolites is discussed below.

### DVS

The mass spectral characteristics of DVS standard were examined for comparison with metabolites. In the LC/MS spectrum of DVS, a protonated molecular ion, [M+H]⁺ was observed at m/z 264. FIG. 7 shows the products of m/z 264 mass spectrum of DVS, obtained from collision induced dissociation (CID), and the proposed fragmentation scheme. Loss of H₂O from the molecular ion yielded the product ion at m/z 246. Further loss of the dimethylamino group yielded the product ion at m/z 201. Loss of the cyclohexanol group from DVS was represented by the product ion at m/z 164. The product ion at m/z 58 was due to (CH₃)₂NCH₂⁺. In addition, the product ions at m/z 107, 133, 145, 159 and 173 corresponded to the methyl, propyl, butyl, pentyl, and hexyl-phenolic portions, respectively, of the DVS molecule. Therefore, these ions could be used to detect sites of metabolism localized to the dimethylamino, hydroxybenzyl, and cyclohexanol groups.

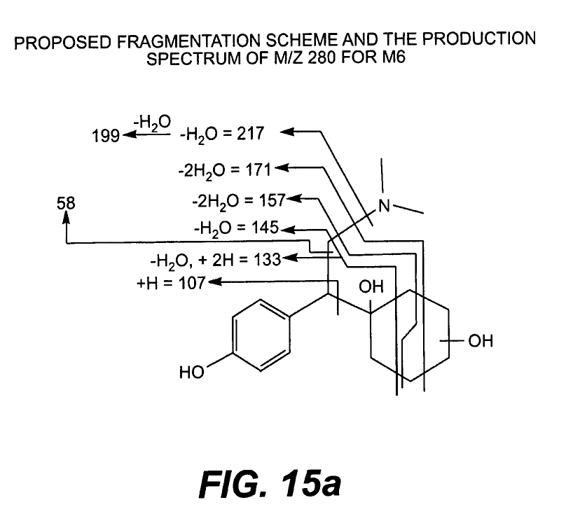

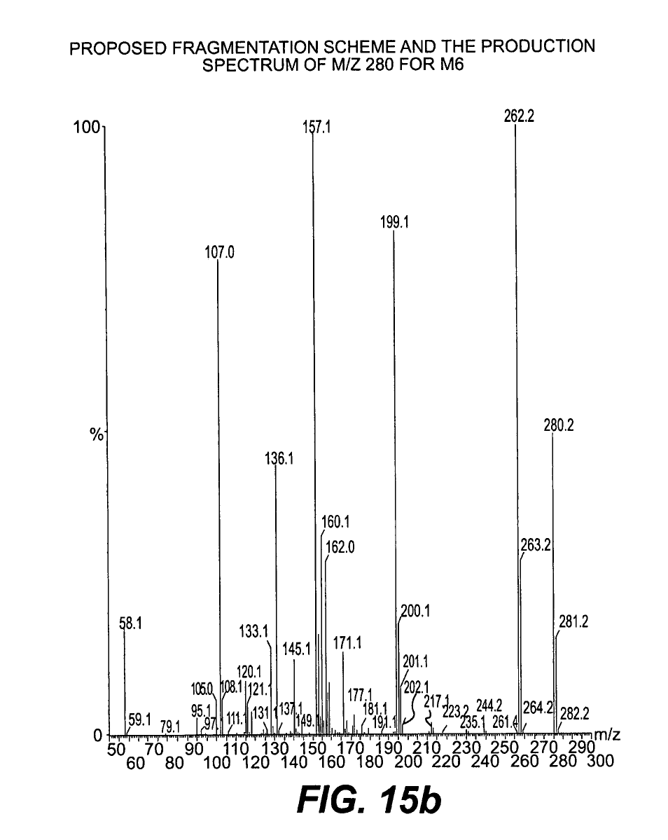

Metabolites M1, M2, M3, M4, M5 and M6 (hydroxy DV compounds) produced a [M+H]⁺ at m/z 280, which was 16 Da larger than DVS and suggested hydroxylation or N-oxidation. FIG. 15 shows the products of m/z 280 spectrum for M6. Mass spectral data for metabolites M1 to M6 were similar. Loss of H₂O from the molecular ion yielded the product ion at m/z 262. The product ions at m/z 58, 107 and 217 for the metabolites versus at m/z 58, 107 and 201 for DVS indicated the cyclohexane ring as the site of metabolism. Therefore, metabolites M1 through M6 were proposed to be hydroxy DV metabolites with the cyclohexane ring as the site of oxidation.

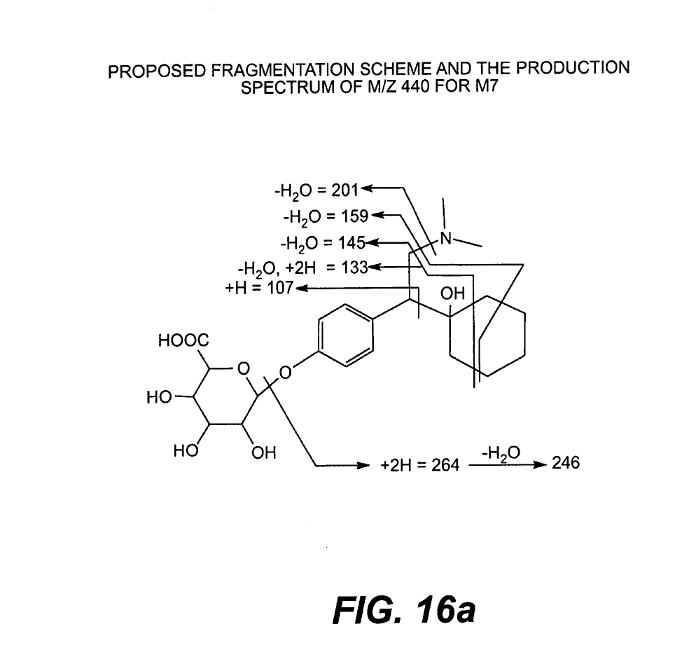

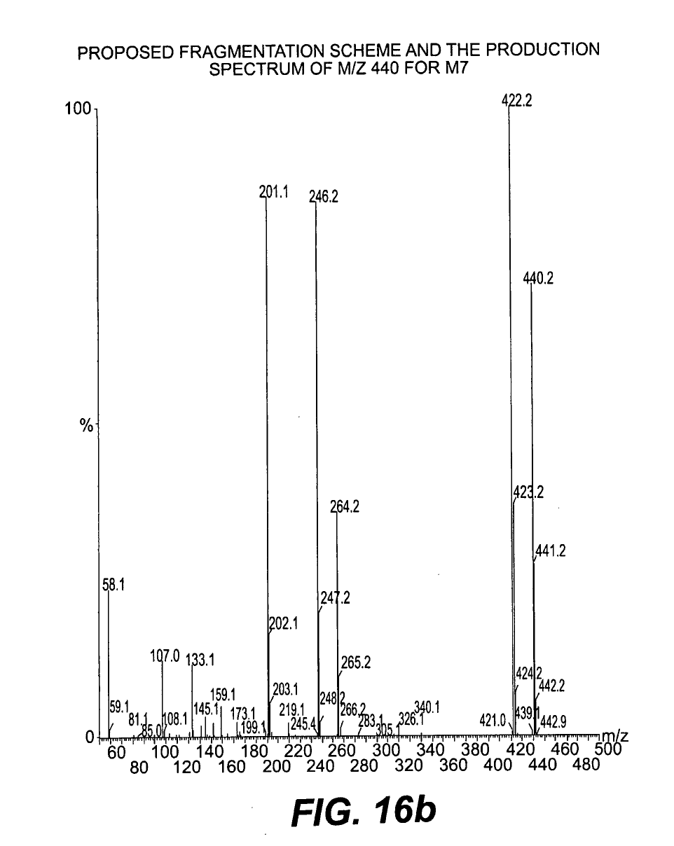

Metabolite M7 (O-desmethylvenlafaxine O-glucuronide, DV glucuronide) The [M+H]⁺ for this metabolite was observed at m/z 440, which indicated a molecular weight of 439. FIG. 16 shows the products of m/z 440 spectrum for M7. The loss of 176 Da from the molecular ion generated the product ion at m/z 264 which indicated that this metabolite was the glucuronide of DVS. The mass spectral data did not indicate the site of conjugation. Incubations performed with dog liver microsomes and DVS or venlafaxine were used to determine the site of glucuronidation. In the presence of only UDPGA, glucuronidation of DVS, but not venlafaxine, was observed. Glucuronidation of venlafaxine was only observed in the presence of both UDPGA and NADPH. The glucuronide that was formed from venlafaxine had the same [M+H]⁺ and retention time as M7, which was the result of O-desmethylation followed by glucuronidation of the phenolic hydroxyl group. The only structural difference between DVS and venlafaxine is that the phenolic hydroxyl group of DVS is methylated on venlafaxine. This showed that a phenol group is required for glucuronidation of DVS-related compounds. Therefore, M7 was proposed to be an O-glucuronide of DV with the phenol group as the site of conjugation.

### Metabolite M9

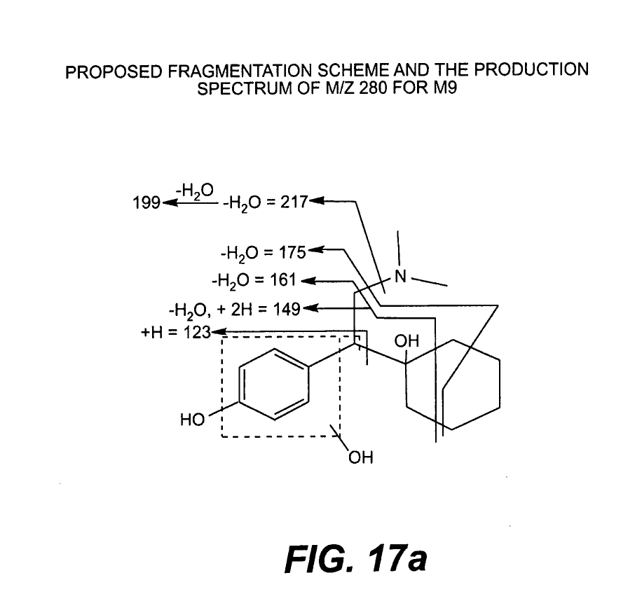

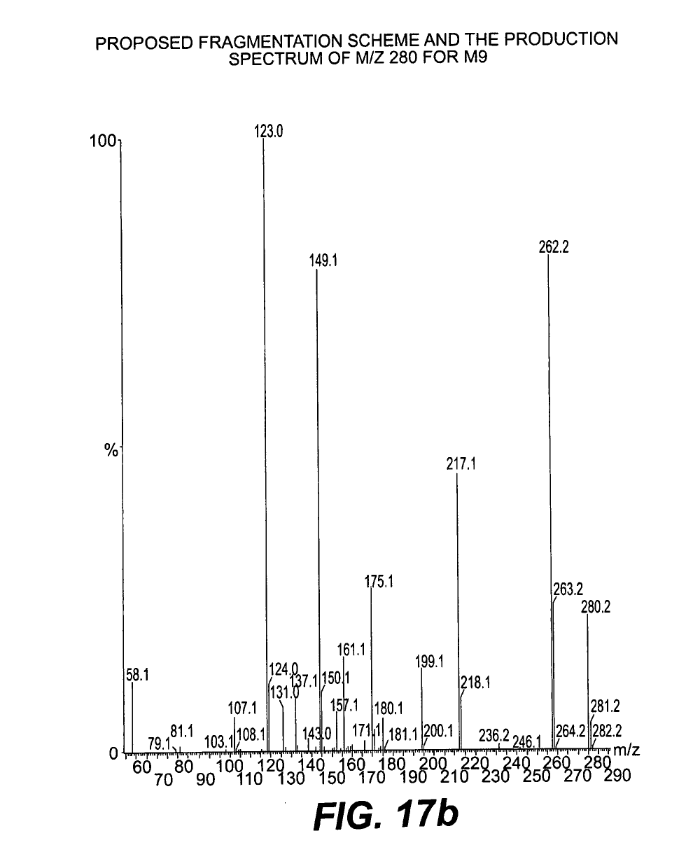

Metabolite M9 produced [M+H]⁺ at m/z 280, which was 16 Da larger than DVS and suggested hydroxylation or N-oxidation. FIG. 17 shows the products of m/z spectrum for M9. The product ions at m/z 123, 149, and 161 were 16 Da higher than the corresponding DVS product ions at m/z 107, 133 and 145, respectively, which indicated hydroxylation of the benzyl group. Therefore, M9 was a hydroxy DV with the benzyl group as the site of oxidation.

### Metabolite M10

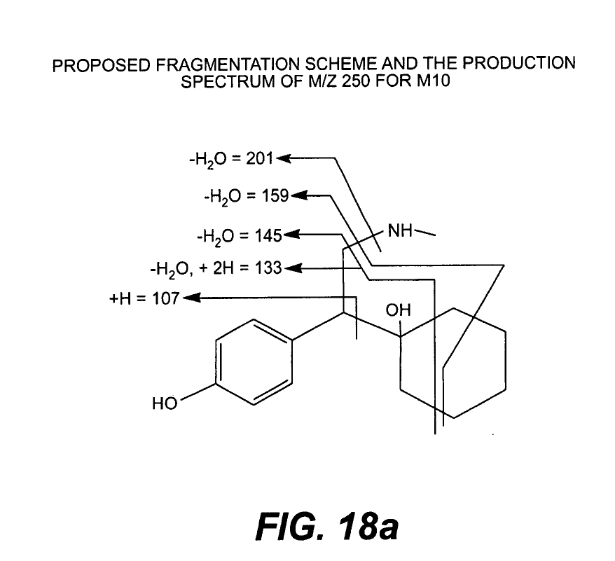

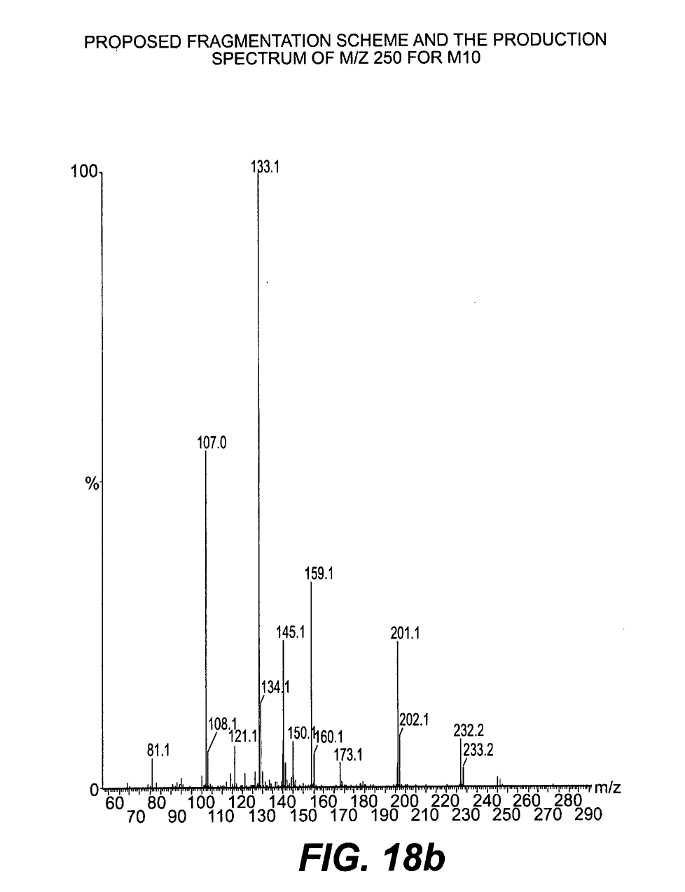

The [M+H]⁺ for M10 was observed at m/z 250. FIG. 18 shows the products of m/z 250 spectrum for M10. Loss of H₂O from the molecular ion at m/z 250 yielded the diagnostic product ion at m/z 232. Subsequent loss of methylamine from m/z 232 generated the product ion at m/z 201. This, and the lack of a product ion at m/z 58, indicated that the dimethylamino group of DV had been converted to a methylamino group by N-demethylation. In addition, the products of m/z 250 mass spectrum for M10 matched the products of m/z 250 mass spectrum for synthetic N,O-didesmethylvenlafaxine. Therefore, M10 was identified as N,O-didesmethylvenlafaxine.

### Metabolites M11a, M11b, M11c, and M12 (Hydroxy DV Glucuronides)

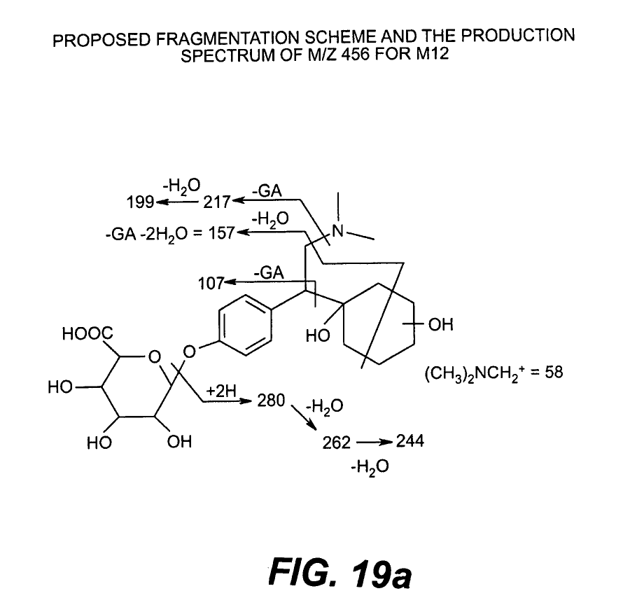

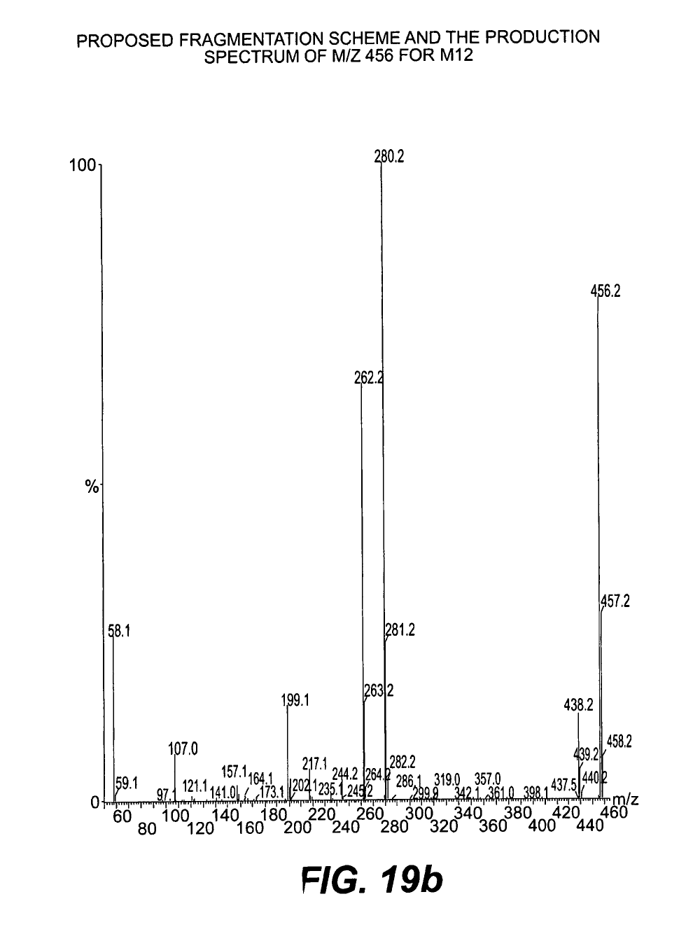

The [M+H]⁺ for M11a, M11b, M11c and M12 were observed at m/z 456, which indicated a molecular weight of 455. FIG. 19 shows the products of m/z 456 spectrum for M12. Mass spectral data for M11a, M11b, M11c and M12 were similar. The loss of 176 Da from the molecular ion yielded the ion at m/z 280, which was the [M+H]⁺ for the hydroxy DV metabolites. The mass spectral data did not indicate the site of conjugation. The phenol group was proposed as the site of conjugation based on the results of in vitro glucuronidation experiments with DVS and venlafaxine discussed for metabolite M7. The product ions at m/z 58, 107 and 217 for the metabolites versus at m/z 58, 107 and 201 for DVS indicated hydroxylation of the cyclohexane ring. Therefore, M11a, M11 b, M11c and M12 were proposed to be O-glucuronides of hydroxy DV metabolites.

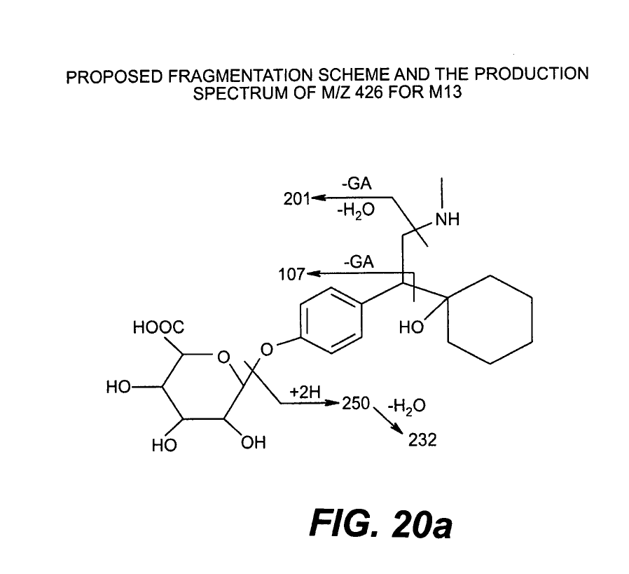

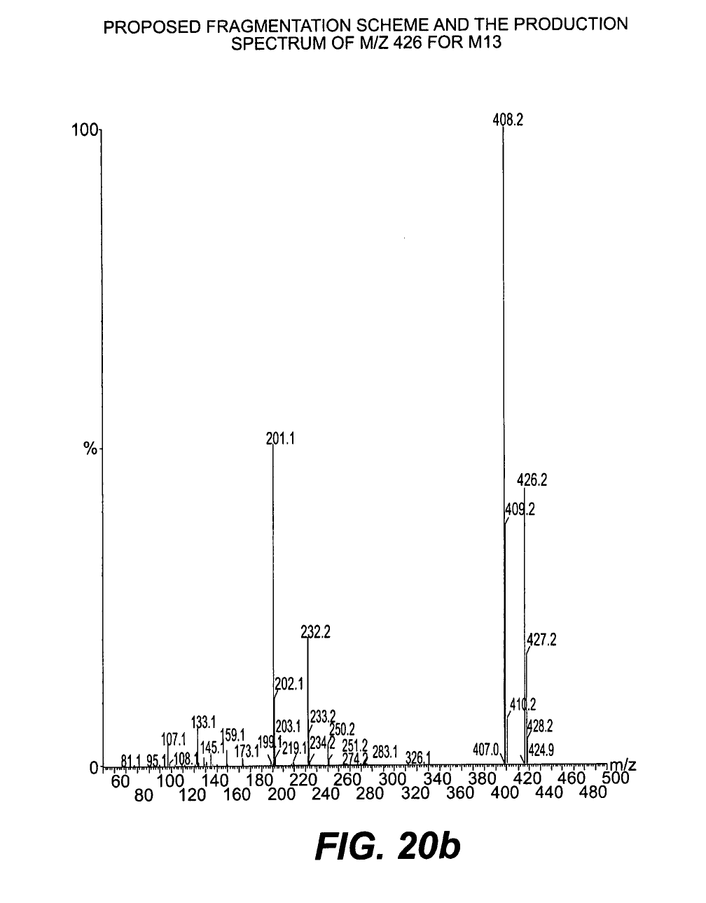

Metabolite M13 (N,O-didesmethylvenlafaxine O-glucuronide). The [M+H]⁺ for this metabolite was observed at m/z 426, which indicated a molecular weight of 425. FIG. 20 shows the product ion spectrum of M13. The loss of 176 Da from m/z 426 yielded the ion at m/z 250. Loss of H₂O from the cyclohexanol moiety yielded the base peak at m/z 408. The loss of 176 Da from the ion at m/z 408 yielded the diagnostic product ion of M10 at m/z 232. Subsequent loss of methylamine from m/z 232 generated the product ion at m/z 201. Therefore, M13 was proposed to be the N,O-didesmethylvenlafaxine O-glucuronide with the phenol group as the site of glucuronidation.

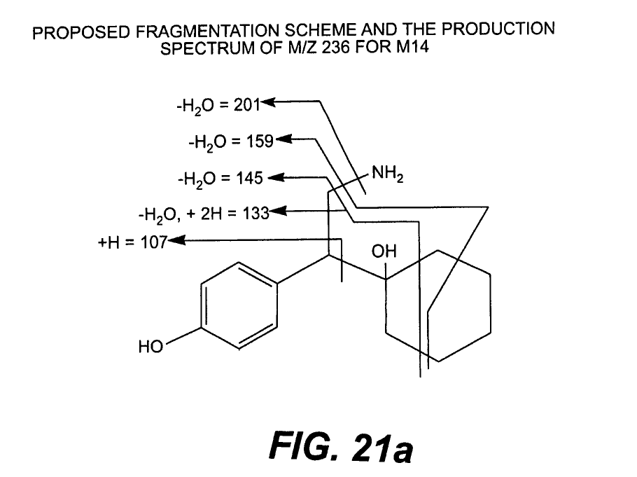

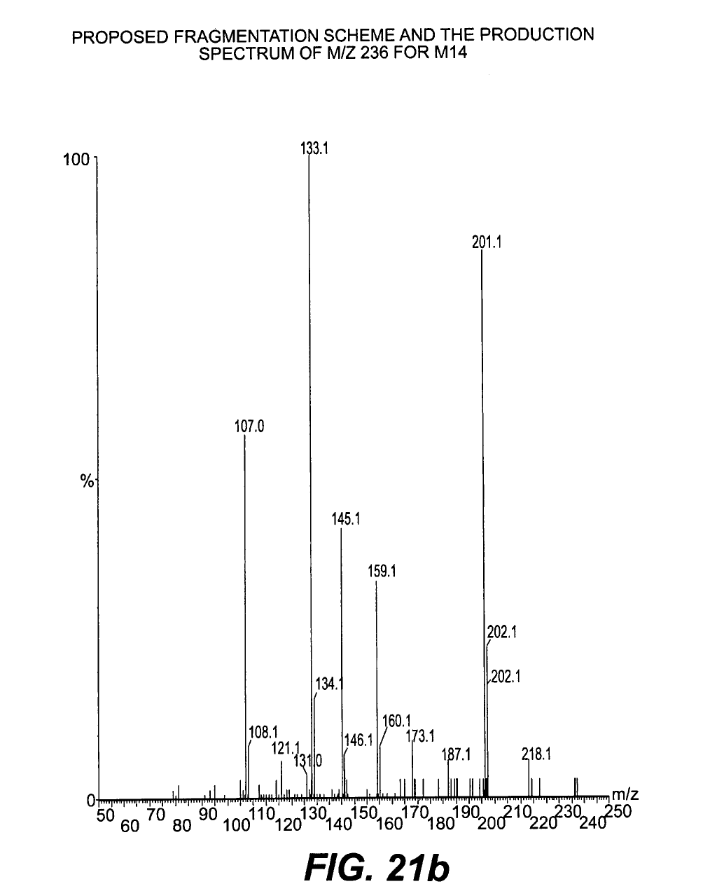

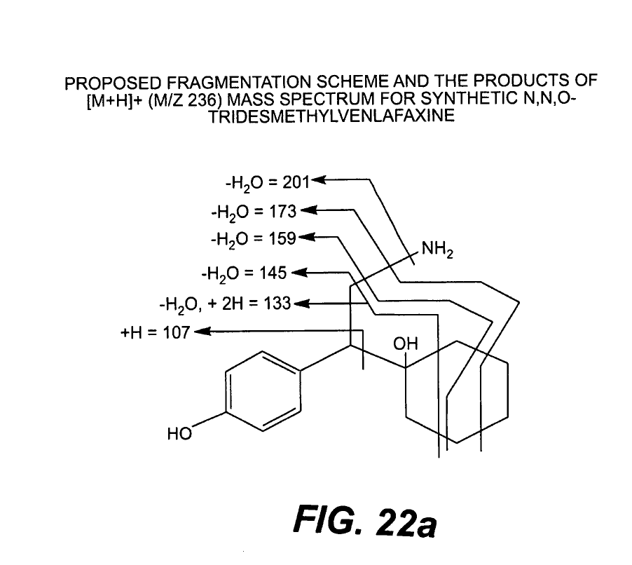

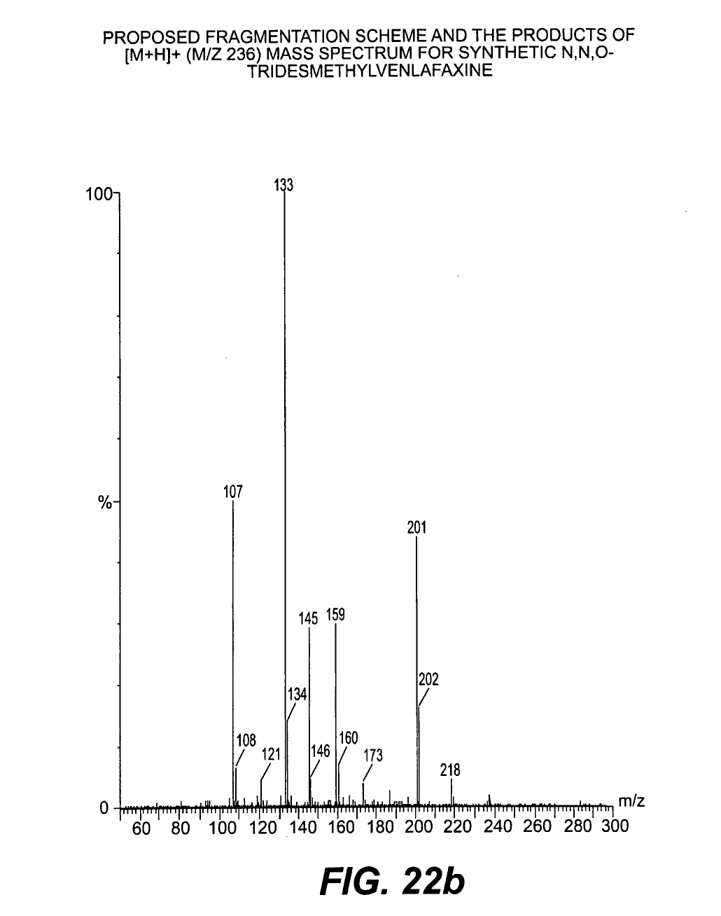

Metabolite M14 produced [M+H]⁺ at m/z 236. FIG. 21 shows the products of m/z 236 spectrum for M14. Loss of H₂O and NH₃ from the molecular ion yielded the product ion at m/z 201. This and the lack of a product ion at m/z 58 indicated N-didemethylation. The product ions at m/z 107, 133, 145, 159 and 173 were also observed for DVS. The products of m/z 236 mass spectrum for M14 matched the mass spectrum of synthetic N,N,O-tridesmethylvenlafaxine, shown in FIG. 22. Therefore, M14 was identified as N,N,O-tridesmethylvenlafaxine.

### N-Oxide DV

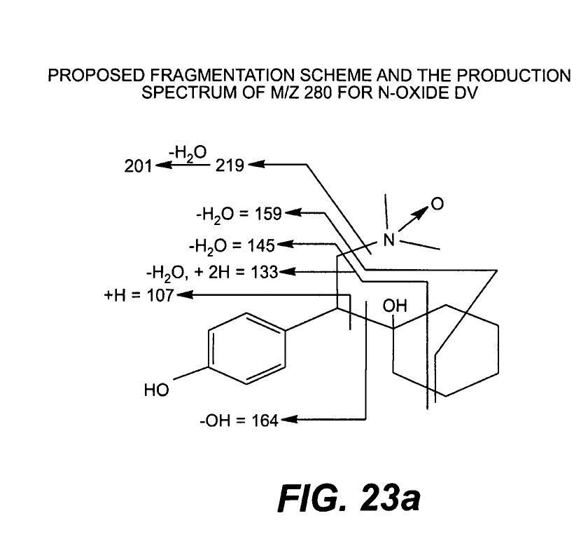

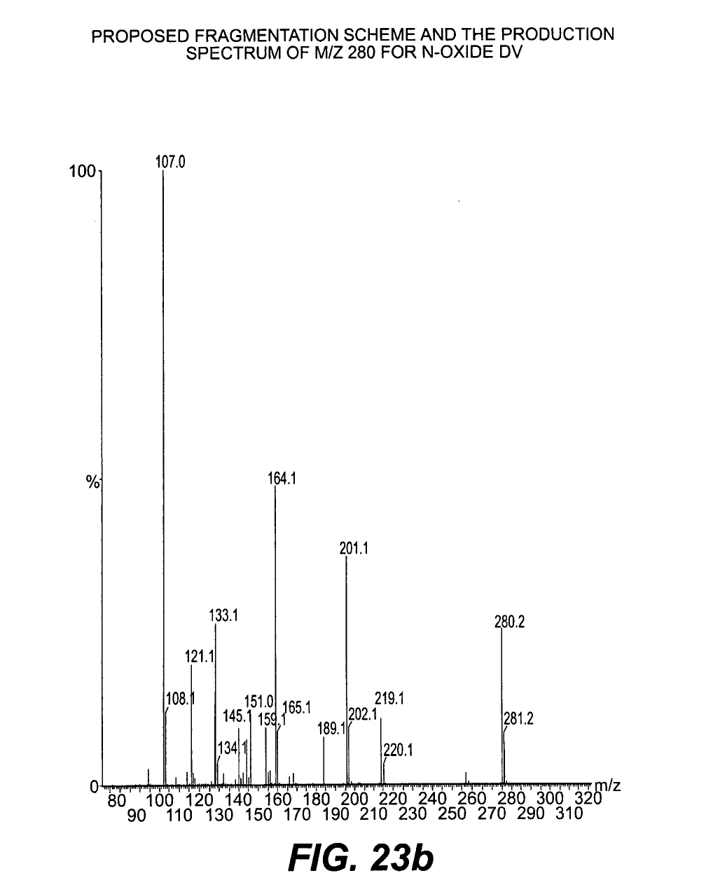

The [M+H]⁺ for this DVS related component was observed at m/z 280, which indicated hydroxylation or N-oxidation. FIG. 23 shows the products of m/z 280 mass spectrum for this DVS related compound. Loss of 61 Da from [M+H]⁺ ion yielded the product ion at m/z 219. This corresponded to loss of dimethylhydroxyamine consistent with an N-oxide. Therefore, this metabolite was identified as N-oxide DV.

### Example 3

### Synthesis of 2-Hydroxy-DV Compounds

The 2-hydroxy-DV compounds of the invention may be produced using the following method. 4-(Dimethylcarbamoylmethyl)phenol in dimethylformamide

(DMF) is treated with K₂CO₃ followed by benzyl bromide. The mixture is stirred at room temperature followed by heating at 60° C. for 1 hour. The mixture is concentrated to remove DMF, diluted with EtOAc and washed with water. Dry MgSO₄ is added, the mixture filtered and concentrated to low volume. Hexane is added to precipitate the ketal intermediate product. Solids are collected via filtration and dryed.

A solution of the 2-benzyloxy-cyclohexanone in 100 mL THF/50 mL MeOH is treated with acid (e.g., HCl), then stirred at room temperature. The reaction is quenched with saturated K₂CO₃, extracted with EtOAc and concentrated to an oil. Product is crystallized from hot EtOAc/hexanes to provide the ketone intermediate as shown in FIG. 2.

A solution of the ketone in THF was added to a suspension of lithium aluminum hydride (LAH) pellets in THF at −78° C. The mixture is warmed to room temperature and stirred for at least 3 hours. The reaction is quenched with MeOH followed by 10% NaOH and stirred for at least 3 hours. The solid are removed by filtration, followed by a wash (e.g., with THF), and concentrated to give a solid. The resulting solid is recrystallized from EtOAc/hexanes to provide the corresponding benzyl ether.

Both benzyl protecting groups may be removed by stirring with Pd/C in 100 mL of ethanol, and hydrogenating under pressure overnight. The solid is purified by filtration followed by an ethanol wash. Solid is concentrated and crystallized from EtOAc/hexane to give the final product.

### Example 4

### Synthesis of 2-Hydroxy DV Glucuronide Compounds

The 2-hydroxy DV glucoronide compounds may be synthesized as follows. To a solution of 2-hydroxy DV (1.0 g, 3.6 mmol) and 2.05 g (4.3 mmol) of the trichloroimidate in methylene chloride (15 mL) is added BF₃OET₂ (0.54 mL, 4.4 mmol) dropwise over a 5 min period. The reaction is stirred overnight under nitrogen atmosphere. Then the reaction mixture is poured into NaHCO₃ (sat) and extracted with methylene chloride. The organic layer is separated, dried and concentrated in vacuo. The crude residue is passed through a short silica column, elution with methylene chloride-methanol. The filtrate is concentrated to provide the protected 2-hydroxy DVO-glucuronide (see FIG. 3).

The protected 2-hydroxy DV glucuronide (the tri acetyl methyl ester) (1.0 g, 1.7 mmol) is taken up in a mixture of dioxane-MeOH—H₂O (2:1:1) 8 mL and LiOH (0.4 g, 17 mmol) is added and the resulting solution is heated to 60° C. for 1 hr. The reaction mixture is then cooled and diluted with acetic acid. The mixture is concentrated in vacuo and the residue may be purified on silica with methylene chloride-methanol to provide 2-hydroxy DV glucuronide.

### Example 5

### Synthesis of N-Oxide DV

N-oxide DV was prepared using a chemical synthesis strategy as follows. To prepare N-oxide DV I shown in FIG. 4: ODV (1.0 g, 3.8 mmol) was taken into chloroform (45 mL) and cooled to 0° C. Then MCPBA (0.786 g, 4.56 mmol) in chloroform (15 mL) was added dropwise to the reaction mixture. The reaction was allowed to stir overnight under nitrogen atmosphere. The temperature was allowed to warm to room temperature during this time. Then the reaction mixture was poured onto a basic alumina column (40 g) that was prepacked with chloroform. The reaction mixture was absorbed onto the alumina column then chloroform (150 mL) was passed through the column (no pressure). Next a methanol:chloroform mixture (1:3) was passed through the column to elute out the desired product. The fractions containing the product were concentrated and the resulting solid was dissolved in chloroform and passed through a Celite pad. The filtrate was concentrated to yield the desired N-oxide (1.26 g, >100%) as a white solid. Mp. 171-173° C. ¹H NMR (DMSO-d₆), δ (ppm): 0.68-1.64 (m, 10H), 2.95 (s, 3H), 3.14 (s, 3H), 3.19 (d, J=5.7 Hz, 1H), 3.54 (d, J=12.7 Hz, 1H), 3.89 (dd, J=7.5 Hz and 7.3 Hz, 1H), 6.67 (d, J=8.4 Hz, 2H), 6.98 (d, J=8.4 Hz, 2H), 9.51 (s, 1H); (M+H)⁺ 280; (M−H)⁻ 278; Anal. Calculated for C₁₆H₂₅NO₃: C, 68.79; H, 9.02; N, 5.01; Found: C, 57.64; H, 7.36; N, 3.73; Analytical HPLC (5-95% Acetonitrile/water); 98.4% at 210 nM; 99.3% at 230 nM.

The N-oxide DV II shown in FIG. 4 [the N-oxide of (S)-4-[2-dimethylamino-1-(1-hydroxy-cyclohexyl)-ethyl]-phenol] was prepared as compound 1.

The compound is a white solid (1.03 g, 97.3%). Mp. 175-176° C. ¹H NMR (DMSO-d₆), δ (ppm): 0.68-1.64 (m, 10H), 2.95 (s, 3H), 3.14 (s, 3H), 3.19 (d, J=5.7 Hz, 1H), 3.54 (d, J=12.7 Hz, 1H), 3.89 (dd, J=7.5 Hz and 7.3 Hz, 1H), 6.67 (d, J=8.4 Hz, 2H), 6.98 (d, J=8.4 Hz, 2H), 9.51 (s, 1H); (M+H)⁺ 280; (M−H)⁻ 278; Anal. Calculated for C₁₆H₂₅NO₃: C, 68.79; H, 9.02; N, 5.01; Found C, 60.62; H, 7.84; N, 4.02; Analytical HPLC (5-95% Acetonitrile/water); 98.0% at 210 nM, 99.0% at 230 nM; Optical rotation; −15.49 (corrected for chloroform impurity).

The N-oxide of (R)-4-[2-Dimethylamino-1-(1-hydroxy-cyclohexyl)-ethyl]-phenol (III) was prepared as compounds I and II (see FIG. 4) This N-oxide DV is a white powder (0.88 g, 82.9%). Mp. 181-182° C.; ¹H NMR (DMSO-d₆), δ (ppm): 0.68-1.64 (m, 10H), 2.95 (s, 3H), 3.14 (s, 3H), 3.19 (d, J=5.7 Hz, 1H), 3.54 (d, J=12.7 Hz, 1H), 3.89 (dd, J=7.5 Hz and 7.3 Hz, 1H), 6.67 (d, J=8.4 Hz, 2H), 6.98 (d, J=8.4 Hz, 2H), 9.51 (s, 1H); (M+H)⁺ 280; (M−H)⁻ 278; Anal. Calculated for C16H25NO3: C, 68.79; H, 9.02; N, 5.01; Found: C, 67.10; H, 8.92; N, 4.77; Optical rotation; +19.07 (corrected for chloroform impurity).

### Example 6

### Receptor Binding Studies to Determine Activity

The compounds of the present invention may be tested for biological activity using receptor assay binding studies. These studies have been described in the following publications, and are also available from Novascreen, Hanover, Md. The receptor binding assays that may be used include, but are not limited to: adrenergic α-2A (human) binding assay (D. B. Bylund et al, J Pharmacol & Exp Ther, 245(2):600-607 (1988); J A Totaro et al, Life Sciences, 44:459-467 (1989)); dopamine transporter binding assay (Madras et al, Mol. Pharmacol., 36:518-524; J J Javitch et al, Mol Pharmacol, 26:35-44 (1984)); histamine H1 binding assay (Chang, et al., J Neurochem, 32:1658-1663 (1979); J I Martinez-Mir, et al., Brain Res, 526:322-327 (1990); E E J Haaksma, et al, Pharmacol Ther, 47:73-104 (1990)); imidazoline binding assay (C M Brown et al, Brit. J Pharmacol, 99(4):803-809 (1990); muscarinic M5 (human recombinant) binding assay (N J Buckley et al, Mol Pharmacol, 35:469-476 (1989);); norepinephrine transporter (human recombinant) binding assay (R. Raisman, et al., Eur J Pharmacol, 78:345-351 (1982); S. Z. Raisman, et al, Eur J Pharmacol, 72:423 (1981)); serotonin transporter (human) binding assay (R J D'Amato, et al, J Pharmacol & Exp Ther, 242:364-371 (1987); N L Brown et al, Eur J Pharmacol, 123:161-165 (1986)). The cellular/functional assays include the norepinephrine transport (NET-T) human (A. Galli, et al., J Exp Biol, 198:2197-2212 (1995); and the serotonin transport (Human) assay (D'Amato et al, cited above and N L Brown et al, Eur J Pharmacol, 123:161-165 (1986)). The results may be measured as % inhibition of the receptor.

### Example 7

### In Vivo Efficacy of the Compounds of the Present Invention in Microdialysis Model

The compounds of the present invention may be evaluated in microdialysis studies, for example, in male Sprague-Dawley rats. M T Taber et al, “Differential effects of coadministration of fluoxetine and WAY-100635 on serotonergic neurotransmission in vivo: sensitivity to sequence of injections,” Synapse, 38(1): 17-26 (October 2000). This technique can capture the neurochemical effects of compounds in the brains of freely-moving rodents. The effects may be studied in the rat dorsal lateral frontal cortex, a brain region thought to be involved in etiology and/or treatment of depression. To see whether any effects on serotonin could be observed, a compound of the present invention (at a dose of 30 mg/kg, sc) may be tested in combination with the selective 5-HT1A antagonist, N-[2-[4-(2-methoxyphenyl)-1-piperazinyl]ethyl]-N-(2-pyridinyl)cyclohexanecarboxamide. The rationale for doing this is to block the somatodendritic 5-HT1A autoreceptors regulating 5-HT release. This eliminates the need to perform a chronic (14 day) neurochemical study with the compound alone to desensitize the 5-HT1A receptors. The conditions of a suitable study are listed below:

Animal: Male Sprague-Dawley rats (280-350 g)

### Brain Region Dorsal Lateral (DL) Frontal Cortex (A/P+3.2 mm, M/L±3.5 mm, DN—1.5 mm)

### Administration:

24 hr post-operative recovery
3 hr equilibration after probe insertion
1 hr 40 min baseline
5-HT_{1A} antagonist N-[2-[4-(2-methoxyphenyl)-1-piperazinyl]ethyl]-N-(2-pyridinyl)cyclohexanecarboxamide (0.3 mg/kg, s.c.) given 20 min before 1-[2-dimethylamino-1-(4-phenol)ethyl]-cis-1,4-cyclohexandiol (30 mg/kg, po)
   Sample Collection Samples collected for 3 hr 2 min post-injections
   Analysis: 5-HT levels quantified by HPLC-ECD

Under these conditions, in vivo neurochemical effects may be observed. The in vivo neurochemical effects of combinations of other SNRIs and SSRIs, like venlafaxine and fluoxetine, with 5-HT1A antagonism may be observed for comparison.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

### Example 8

### Additional Metabolites of Desvenlafaxine

Additional embodiments of the instant invention include the following desvenlafaxine metabolites:

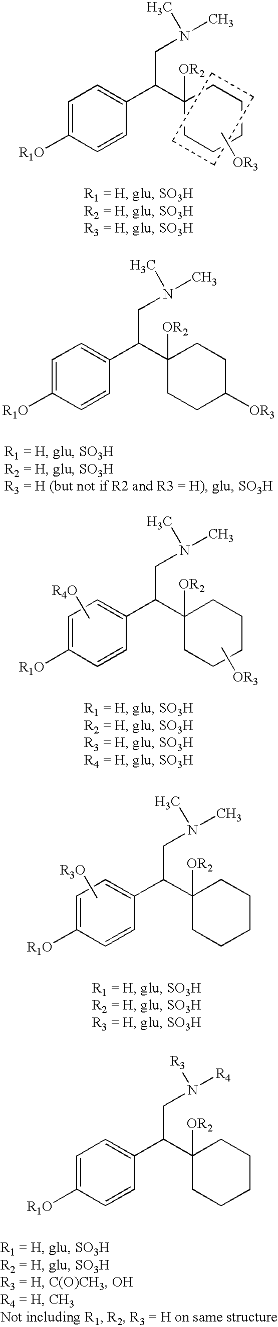

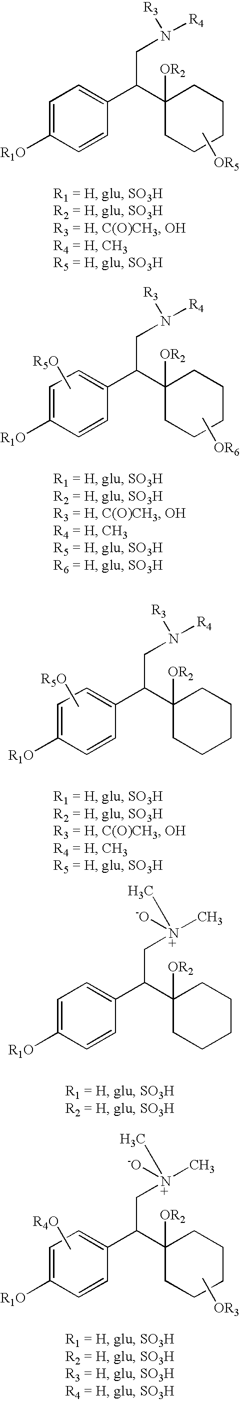

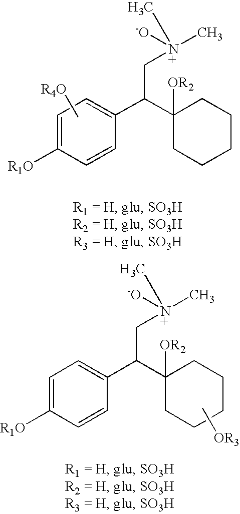

We claim:

1. An isolated DV metabolite or derivative of the formula 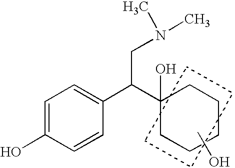
wherein
a hydroxy group is attached to one 2-position or 3-position carbon on the cyclohexyl ring;
and pharmaceutically acceptable salts thereof.

2. The isolated DV metabolite or derivative of claim 1, wherein the hydroxy group is attached to the 2-position carbon on the cyclohexyl ring.

3. The isolated DV metabolite or derivative of claim 1, wherein the hydroxy group is attached to the 3-position carbon on the cyclohexyl ring.

4. An isolated DV metabolite or derivative of the formula 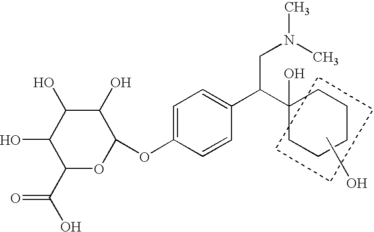
wherein a hydroxy group is attached to one 2-position, 3-position, or 4-position carbon on the cyclohexyl ring;
and pharmaceutically acceptable salts thereof.

5. The isolated DV metabolite of claim 4, wherein the hydroxy group is attached to the 2-position carbon on the cyclohexyl ring.

6. The isolated DV metabolite of claim 4, wherein the hydroxy group is attached to the 3-position carbon on the cyclohexyl ring.

7. The isolated DV metabolite of claim 4, wherein the hydroxy group is attached to the 4-position carbon on the cyclohexyl ring.

8. An isolated DV metabolite or derivative of the formula 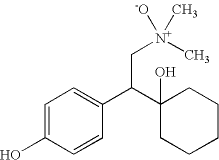
and pharmaceutically acceptable salts thereof.

9. An isolated DV metabolite or derivative of the formula 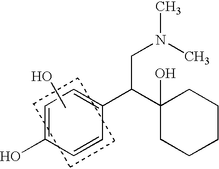
wherein a hydroxy group is attached to one 2-position or 3-position carbon on the benzyl;
and pharmaceutically acceptable salts thereof.

10. The isolated DV metabolite of claim 9, wherein the hydroxy group is attached to the 2-position carbon on the benzyl.

11. The isolated DV metabolite of claim 9, wherein the hydroxy group is attached to the 3-position carbon on the benzyl.

12. A pharmaceutical composition comprising a compound of claim 1, claim 4, claim 8, or claim 9 and a pharmaceutically acceptable carrier or excipient.

13. The pharmaceutical composition of claim 12 further comprising, one or more of venlafaxine, O-desmethylvenlafaxine, and O-desmethylvenlafaxine succinate, or their pharmaceutically acceptable salts.

14. A method of treating at least one central nervous system disorder in a mammal comprising providing to a mammal in need thereof an effective amount of a compound of claim 1, claim 4, claim 8, or claim 9.

15. The method of claim 14, wherein the compound is administered orally.

16. An isolated DV metabolite or derivative chosen from: 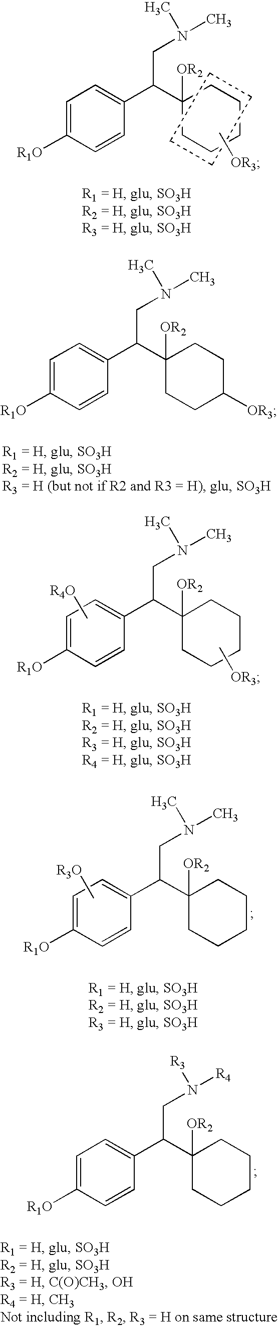 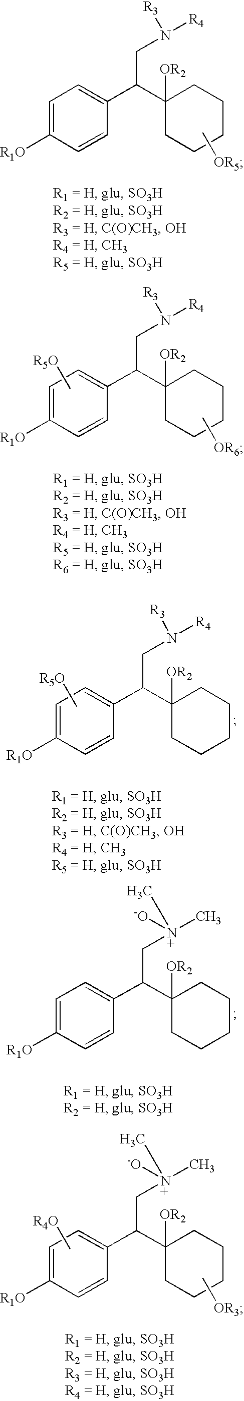 [chemical structure]
